(19) United States
(12) Patent Application Publication
(10) Pub. No.: US 2025/0108096 A1
(43) Pub. Date: Apr. 3, 2025

(54) ALCOHOL CLEANSING METHODS

(71) Applicant: Aperiam Bio, Inc., Brooklyn, NY (US)

(72) Inventors: Austin Woodrow Cole, Brooklyn, NY (US); Guillaume Cottarel, Durham, NC (US)

(73) Assignee: Aperiam Bio, Inc., Brooklyn, NY (US)

(21) Appl. No.: 18/900,033

(22) Filed: Sep. 27, 2024

Related U.S. Application Data
(60) Provisional application No. 63/541,239, filed on Sep. 28, 2023.

(51) Int. Cl. A61K 38/44; A61K 9/00; A61P 39/02

(52) U.S. Cl. CPC A61K 38/443; A61K 9/007; A61P 39/02

(57) ABSTRACT

The present disclosure generally relates to technologies for cleansing at least a portion of a lung of a subject (e.g., from at least one alcoholic irritant) and methods of freshening the breath of a subject. Such technologies comprise introducing, to at least a portion of a lung of a subject, a composition comprising at least one enzyme known to enzymatically break down at least one alcoholic irritant present systemically in the subject, including in at least a portion of said lung.

### CROSS-REFERENCE TO RELATED APPLICATIONS

This application claims the benefit under 35 U.S.C. § 119 (e) of U.S. Provisional Patent Application No. 63/541,239, filed Sep. 28, 2023, the entire contents of which is incorporated herein by reference in its entirety.

### BACKGROUND

Alcoholic irritants (e.g., ethanol, methanol) are found in numerous products, such as alcoholic beverages and household products (e.g., cleaning products). Exposures to alcoholic irritants, in particular, systemic exposures at high-levels and/or for prolonged periods of time, can be harmful to subjects (e.g., humans).

As alcoholic irritants, such as ethanol, are highly diffusible through cell membranes and are metabolized in a plurality of tissues, alcoholic irritants can affect most organs. For example, alcoholic irritant consumption (e.g., an alcoholic beverage), can result in disinhibition, nausea, vomiting, memory loss, seizures, slowed breathing, impaired speaking and/or coordinating, unstable gate, coma, and even death. Further, renal failure is common after ethylene glycol and diethylene glycol ingestion, whereas optic neuritis and visual impairment have been observed because of methanol ingestion.

According to the Center for Disease Control, household products, such as cleaning agents, personal care and topical products, and pesticides, which often comprise alcoholic irritants, are among the top ten substances responsible for poisoning exposures annually. Further, and according to the 2021 National Survey on Drug Use and Health (NSDUH), 219.2 million people ages 12 and older drink alcohol at some point in their lifetime. 29.5 million people ages twelve or older in the United States are affected by Alcohol Use Disorder (AUD).

US20220047682A1 describes an amino acid sequence encoding ADH/KRED bound to at least one long-acting molecule or complexing molecule. The long-acting alcohol dehydrogenase disclosed is described as having extended circulatory half-lives, higher area under the curve value, lower clearance value, lower elimination rate and higher t½ measure within the blood and serum compared to wild-type ADH.

In view the high frequency of consumption of alcoholic irritants, and potentially harmful effects of such consumption, a need remains for novel compositions and methods to remove and/or degrade (e.g., cleanse) alcoholic irritants in vivo in subjects.

### SUMMARY OF THE INVENTION

The present disclosure provides, among other things, methods for cleansing (e.g., degrading, removing) from at least a portion of a lung of a subject at least one alcoholic irritant, such as ethanol.

In one aspect, the present disclosure provides a method of cleansing at least a portion of a lung of a subject comprising introducing to at least a portion of a lung of a subject a composition comprising at least one enzyme known to enzymatically breakdown at least one alcoholic irritant present systemically in said subject, including in at least a portion of said lung.

In some embodiments, the at least one alcoholic irritant comprises methanol or ethanol. In some embodiments, said subject consumed one or more alcoholic beverages.

In some embodiments, the at least one enzyme comprises alcohol dehydrogenase (ADH), aldehyde dehydrogenase (ALDH), variants thereof, or combinations thereof.

In some embodiments, rate of cleansing the at least one alcoholic irritant from the subject's blood per minute is improved by at least about 1%, 2%, 3%, 4%, 5%, 6%, 7%, 8%, 9%, or 10% compared to a control subject wherein no enzyme known to enzymatically breakdown the least one alcoholic irritant was introduced.

In one aspect, the present disclosure provides, a method of cleansing ethanol from at least a portion of a lung of a subject comprising introducing to at least a portion of a lung of a subject a composition comprising alcohol dehydrogenase (ADH), aldehyde dehydrogenase (ALDH), variants thereof, or combinations thereof.

In some embodiments, said subject consumed one or more alcoholic beverages.

In some embodiments, said composition is introduced before, during, or after the consumption of said one or more alcoholic beverages.

In some embodiments, said composition is introduced immediately before, during, or immediately after the consumption of said one or more alcoholic beverages.

In some embodiments, said composition is introduced shortly before, during, or shortly after the consumption of said one or more alcoholic beverages.

In some embodiments, said subject's blood alcohol level is reduced by 10% or more, 25% or more, 50% or more, 75% or more, or 90% or more about one hour after said introduction compared to said subject's blood alcohol level in an absence of said introduction.

In some embodiments, said subject's blood alcohol level is measured by a blood test or by administering a breathalyzer test.

In some embodiments, rate of cleansing ethanol from the subject's blood per minute is improved by at least about 1%, 2%, 3%, 4%, 5%, 6%, 7%, 8%, 9%, or 10% compared to a control subject wherein no ADH, ALDH, variants thereof, or combinations thereof were introduced.

In one aspect, the present disclosure provides, a method of cleansing an alcoholic irritant from at least a portion of a lung of a subject comprising: (a) introducing to at least a portion of a lung of a subject an effective amount of a composition comprising alcohol dehydrogenase (ADH), aldehyde dehydrogenase (ALDH), human variants thereof, or combinations thereof; and (b) allowing an enzymatic breakdown of at least a portion of any alcoholic irritant that has diffused or migrated from the subject's circulatory system into the subject's lung and lung mucous.

In some embodiments, said subject is contemplating a consumption of, is in a process of consuming, or has consumed one or more solid or liquid preparations comprising an alcoholic irritant. In some embodiments, said preparations comprises a pill, a capsule, a gummy, a sublingual, a lozenge, an injectable, a food, a beverage, or combinations thereof.

In some embodiments, said alcoholic irritant is selected from the group consisting of methanol, ethanol, propanol, isopropyl alcohol, benzylic alcohol, phenylethanol, geraniol, retinol, and ethylene glycol, and combinations thereof.

In some embodiments, said composition further comprises coenzymes, cofactors, co-substrates, or combinations thereof. In some embodiments, said composition further comprises nicotinamide adenine dinucleotide (NAD⁺, NADP⁺), flavin adenine dinucleotide (FAD) for alcohol oxidase, pyrroloquinoline quinone (PQQ), or combinations thereof.

In some embodiments, said composition further comprises NADH oxidase.

In some embodiments, rate of cleansing the alcoholic irritant from the subject's blood per minute is improved by at least about 1%, 2%, 3%, 4%, 5%, 6%, 7%, 8%, 9%, or 10% compared to a control subject wherein no ADH, ALDH, variants thereof, or combinations thereof were introduced.

In some embodiments, rate of cleansing the alcoholic irritant from the subject's blood per minute is improved by at least about 1%, 2%, 3%, 4%, 5%, 6%, 7%, 8%, 9%, or 10% compared to a control subject wherein no ADH, ALDH, NADH oxidase, variants thereof, or combinations thereof were introduced.

In one aspect, the present disclosure provides a method of cleansing ethanol from at least a portion of a lung of a subject comprising introducing to at least a portion of a lung of a subject a composition comprising aldehyde dehydrogenase (ALDH), a variant thereof, or a combination thereof.

In some embodiments, rate of cleansing ethanol from the subject's blood per minute is improved by at least about 1%, 2%, 3%, 4%, 5%, 6%, 7%, 8%, 9%, or 10% compared to a control subject wherein no ALDH, variants thereof, or combinations thereof were introduced.

In some embodiments, compositions of the present disclosure comprise a liquid or a solid. In some embodiments, said composition comprises a mist, an aerosol, a vapor, a drop, a snortable, an inhalable, a vapable, or a powder.

In some embodiments, said enzyme, a variant thereof, or a combination thereof resides substantially in the subject's pulmonary tissue.

In some embodiments, said enzyme, a variant thereof, or a combination thereof is introduced under conditions that inhibit or do not support systemic delivery of said enzyme, a variant thereof, or a combination thereof to said subject.

In some embodiments, said enzyme, a variant thereof, or a combination thereof is introduced to the at least a portion of the lung of said subject in a manner that minimizes a systemic introduction into said subject.

In some embodiments, said enzyme, a variant thereof, or a combination thereof is introduced in a manner that does not include pulmonary transmucosal delivery of said enzyme, a variant thereof, or a combination thereof to said subject, which results in an undesired systemic delivery.

In some embodiments, said enzyme, a variant thereof, or a combination thereof excludes an introduction of an ADH/KRED bound to at least one long-acting molecule or complexing molecule. In still other embodiments, an introduction excludes an introduction via inhalation of an atomized solution of an ADH/KRED bound to at least one long-acting molecule or complexing molecule.

In one aspect, the present disclosure provides method of freshening the breath of a subject comprising introducing to at least a portion of a lung of a subject a composition comprising at least one enzyme known to enzymatically breakdown at least one alcoholic irritant present systemically in said subject, including in at least a portion of said lung.

In one aspect, the present disclosure provides methods of freshening the breath of a subject comprising introducing to at least a portion of a lung of a subject a composition comprising alcohol dehydrogenase (ADH), aldehyde dehydrogenase (ALDH), variants thereof, or combinations thereof.

In one aspect, the present disclosure provides methods of freshening the breath of a subject comprising: (a) introducing to at least a portion of a lung of a subject an effective amount of a composition comprising alcohol dehydrogenase (ADH), aldehyde dehydrogenase (ALDH), human variants thereof, or combinations thereof; and (b) allowing an enzymatic breakdown of at least a portion of any alcoholic irritant that has diffused or migrated from the subject's circulatory system into the subject's lung and/or lung mucous.

In one aspect, the present disclosure provides methods of freshening the breath of a subject comprising introducing to at least a portion of a lung of a subject a composition comprising aldehyde dehydrogenase (ALDH), a variant thereof, or a combination thereof.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

FIG. 1 is a graph illustrating circulating ethanol concentration after ethanol administration by oral gavage alone, or in combination with pulmonary administration of ADH, in mice.

FIG. 2 provides a study protocol to evaluate the effect of pulmonary administration of ADH or ADH and NAD⁺ on circulating ethanol levels in mice after acute ethanol intoxication.

FIG. 3 is a graph illustrating blood alcohol concentration (mmol) over time to 240 minutes (4 hours) after ethanol administration by oral gavage alone, or in combination with pulmonary administration of either (i) ADH or (ii) ADH and NAD⁺15 minutes after ethanol oral gavage, in mice.

FIG. 4 is a graph illustrating alcohol clearance in the blood is accelerated in mice by pulmonary administration of ADH and NAD (light grey) after acute ethanol intoxication compared to control (dark grey, no enzyme).

FIG. 5 is a graph illustrating blood ethanol concentration (mmol) over time to 4 hours after no alcohol administration, alcohol administration by oral gavage alone (no enzyme), or alcohol administration by oral gavage in combination with pulmonary administration of either (i) ADH or (ii) ADH and NAD (1 mg ADH; 0.15 mmol NAD) 15 minutes after alcohol oral gavage, in mice.

FIG. 6 is a graph showing the blood ethanol level in mice administered either (i) ADH and NAD; (ii) ADH, NAD, and ALDH; or (iii) ADH and NAD after acute ethanol intoxication. Data are the average of two measurements per group (2 mice per group). Zero and Pre60 were the injection timepoints of alcohol in the blood of mice and the delivery of the enzyme solutions into the lungs, respectively. Every 10 minutes after enzyme solution delivery, blood was drawn, and alcohol levels were measured.

FIG. 7 is a graph showing the blood ethanol level in mice administered either (i) ADH and NAD; or (ii) ADH, NAD, and a variant ALDH after acute ethanol intoxication. Data are the average of two measurements per group (2 mice per group). Zero and Pre60 were the injection timepoints of alcohol in the blood of mice and the delivery of the enzyme solutions into the lungs, respectively. Every 15 minutes after enzyme solution delivery, blood was drawn, and alcohol levels were measured.

FIG. 8 is a graph showing the blood ethanol level in mice administered either (i) no enzyme; (ii) ADH; (iii) ADH and a variant ALDH; or (iv) ADH, a variant ALDH, and a variant NOX after acute ethanol intoxication. Data are the average of two measurements per group (2 mice per group). Zero and Pre60 are the injection timepoints of alcohol in the blood of mice and the delivery of the enzyme solutions into the lungs, respectively. Every 15 minutes after enzyme solution delivery, blood was drawn, and alcohol levels were measured.

FIG. 9 is a graph showing the milligrams per liter of ethanol cleansed from human blood each minute following either (i) ethanol consumption (control; no enzyme in human lung); or (ii) ethanol consumption and subsequent inhalation of a nebulized solution comprising 100 mg alcohol dehydrogenase, 800 mg aldehyde dehydrogenase, and 100 mg NADH oxidase (enzyme in human lung).

### DETAILED DESCRIPTION

Consumption of alcoholic irritants (e.g., ethanol, methanol) can result in adverse effects (e.g., nausea, vomiting) and is a public health concern. According to the Center for Disease Control, household products, such as cleaning agents, personal care and topical products, and pesticides, which often comprise alcoholic irritants, are among the top ten substances responsible for poisoning exposures annually. Further, and according to the 2021 National Survey on Drug Use and Health (NSDUH), 219.2 million people ages 12 and older drink alcohol at some point in their lifetime. 29.5 million people ages twelve or older in the United States are affected by alcohol use disorder.

In view the high frequency of consumption of alcoholic irritants, and potentially harmful effects of such consumption, a need remains for novel compositions and methods to remove and/or degrade (e.g., cleanse) alcoholic irritants in vivo in subjects.

The present disclosure generally relates to, technologies for cleansing from at least a portion of a lung of a subject at least one alcoholic irritant. Such technologies comprise introducing, to at least a portion of a lung of a subject, a composition comprising at least one enzyme known to enzymatically break down at least one alcoholic irritant present systemically in the subject, including in at least a portion of said lung (e.g., to reduce the level of alcoholic irritant in the subject, such as reducing the level of alcoholic irritant in the blood of the subject).

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. Although not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety. In case of conflict, the present specification, including definitions, will control. Other aspects are set forth within the claims that follow.

The practice of the present technology will employ, unless otherwise indicated, conventional techniques of tissue culture, immunology, molecular biology, microbiology, chemical engineering, and cell biology, which are within the skill of the art.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or combinations thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations that can be varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.”

### Definitions

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. The terms or “acceptable,” “effective,” or “sufficient” when used to describe the selection of any components, ranges, dose forms, etc. disclosed herein intend that said component, range, dose form, etc. is suitable for the disclosed purpose.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, the term “adverse event” or “AE”, refers to any undesirable medical occurrence in a subject that consumed an alcoholic irritant. An adverse event can therefore be any unfavorable and/or unintended sign (e.g., an abnormal laboratory finding), symptom (e.g., nausea, vomiting), and/or condition temporally associated with the consumption of the alcoholic irritant.

As used herein, the term “agent” refers to a compound or entity of any chemical class including, for example, polypeptides, polynucleotides, saccharides, lipids, small molecules, or combinations thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature.

As used herein, the term “alcoholic beverage” refers to a drink comprising any fermented liquor, such as wine, beer, or distilled spirits that comprises ethanol.

As used herein, the term “alcoholic irritant” refers to an identifiable, isolatable substance comprising at least one hydroxyl group (—OH) that is freely circulating within a subject via a circulatory system and capable of diffusing between lung tissue and the circulatory system of a subject. Alcoholic irritants can include, for example, primary alcohols (e.g., ethanol, benzylic alcohol, phenylethanol, geraniol and retinol) and secondary alcohols (e.g., 2-propanol, 2-butanol). Alcoholic irritants can also include, for example, straight- or branched-chain lower aliphatic alcohols (e.g., methanol, ethanol, propanol, isopropyl alcohol and the like). Further examples of alcoholic irritants include, without limitation, diethylene glycol and ethylene glycol.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, use of the term “consumed” or “consumption” in connection with an alcoholic irritant being “consumed” by a subject, refers to any method in which an alcoholic irritant enters the body of the subject. An alcoholic irritant may be “consumed” by a plurality of methods, including, for example, snorting, inhaling, sublingual absorption, enema, vaginal absorption, eyeballing, injection, and ingestion.

As used herein, “optional” or “optionally” means that the subsequently described event or circumstance can or cannot occur, and that the description includes instances where the event or circumstance occurs and instances where it does not.

As used herein, “introducing” or “administering” or the “administration” of an agent (e.g., a prescription or non-prescription agent, a dietary supplement, a food, or a cosmetic agent) or compound/product (including a composition (i.e., a formulation or cosmetic)) to at least a portion of a lung of a subject includes any route of introducing or delivering to a subject an agent/compound/product to perform its intended function. Introduction into at least a portion of a lung of a subject of an enzyme agent and/or its cofactors and/or enzymes for co-factor regeneration may be carried out by any suitable route but under conditions that do not support a significant or substantial permeation of the agent beyond pulmonary tissue (i.e., minimization of systemic introduction). A typical introduction step can be carried out by inhalation. However, to an extent significant or substantial systemic introduction is to be avoided, inhalation of an atomized solution of enzyme and/or co-enzyme and/or co-factors and/or enzymes for co-factor regeneration is preferably avoided. Avoiding or minimization of systemic introduction means that a majority of the agent introduced continues to reside in pulmonary tissue. In certain embodiments of this disclosure, less than about 50%, less than about 40%, less than about 30%, less than about 20%, or less than about 10% of the agent introduced permeates mucosal tissue (i.e., introduction is not transmucosal) into a subject's blood or serum. Introduction can also be carried out intratracheally, the agent(s) introduced eventually finding their way into at least a portion of a lung of a subject. Alternatively, administration may be carried out topically, intranasally, but preferably not intraperitoneally, intradermally, ophthalmically, intrathecally, intracerebroventricularly, iontophoretically, transmucosally, intravitreally, or intramuscularly. Introduction includes self-introduction, the introduction by another or introduction by use of a device (e.g., a vaper, a dry powder inhaler or vapor pump, but preferably not an infusion pump). In one embodiment of the disclosure, introduction excludes transmucosal delivery of an agent through at least a portion of a lung of a subject. In another embodiment, introduction excludes inhalation of an atomized solution containing ADH/KRED bound to at least one long-acting molecule or complexing molecule. Thus, the conditions of the introduction step are intended to minimize, if not inhibit, a systemic delivery of an agent.

As used herein, to “ameliorate” or “ameliorating” a condition (e.g., consumption of an alcoholic irritant) refers to results that, in a statistical sample or specific subject, make the occurrence of the condition (or a sign or symptom thereof) better or more tolerable in a sample or subject to which an agent of the disclosure is introduced relative to a control sample or subject.

As used herein, the term “amino acid” includes both a naturally occurring amino acid and a non-natural amino acid. The term “amino acid,” unless otherwise indicated, includes both isolated amino acid molecules (i.e., molecules that include both, an amino-attached hydrogen and a carbonyl carbon-attached hydroxyl) and residues of amino acids (i.e., molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H-Ala-OH or to any one of the alanine residues H-Ala-, -Ala-OH, or -Ala-. Unless otherwise indicated, all amino acids found in the compounds (e.g., enzymes) described herein can be either in D or L configuration. An amino acid that is in D configuration may be written such that “D” precedes the amino acid abbreviation. For example, “D-Arg” represents arginine in the D configuration. According to convention, if there is no “D” or “L” that precedes the amino acid, the amino acid is assumed to be of the “L” configuration. Notably, many amino acid residues are commercially available in both D- and L-form.

The term “amino acid” includes salts thereof, including physiologically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH. Amino acid protecting groups are well known in the art. A comprehensive review of amino acid protecting groups can be found in: Isidro-Llobet et al., Chem. Rev. (2009) 109:2455-2504.

With the exception of the N-terminal amino acid, all abbreviations of amino acids (for example, Phe) in this disclosure stand for the structure of —NH—C(R)(R′)—CO—, wherein R and R′ each is, independently, hydrogen or the side chain of an amino acid (e.g., R-benzyl and R′═H for Phe). Accordingly, phenylalanine is H-Phe-OH. The designation “OH” for these amino acids, or for peptides (e.g., Lys-Val-Leu-OH) indicates that the C-terminus is the free acid. The designation “NH₂” in, for example, H-Phe-D-Arg-Phe-Lys-NH₂ indicates that the C-terminus of the protected peptide fragment is amidated. In each case, an “H” preceding an amino acid or peptide indicates that the amine of the amino acid or peptide N-terminus is unmodified (i.e. is —NH₂). Further, certain R and R′, separately, or in combination as a ring structure, can include functional groups that may require protection during the liquid phase or solid phase synthesis.

As used herein the terms “carrier” or “pharmaceutically acceptable carrier” or “physiologically acceptable carrier or “cosmetically acceptable carrier” refer to a diluent, adjuvant, excipient, or vehicle with which an agent (e.g., dietary supplement, food, or cosmetic agent)/product/composition (including a formulation or cosmetic) is administered/introduced or formulated for administration/introduction. Non-limiting examples of such acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable carriers are described in Remington's Pharmaceutical Sciences by E. W. Martin, herein incorporated by reference in its entirety.

As used herein, the term “cleansing” refers to the degradation and/or removal of substrates (e.g., alcoholic irritants) in/from a subject. Such reactions can be catalyzed by an enzyme (e.g., an enzyme known to enzymatically breakdown at least one alcoholic irritant) and can reduce the level of a substrate (e.g., alcoholic irritant) present in a subject (e.g., in at least a portion of a lung of the subject, in the systemic circulation of the subject).

As used herein, the term “co-factor” refers to a non-protein chemical compound required for the activity of an enzyme as a catalyst. Co-factors can be divided into two groups: inorganic ions and organic molecules. Organic molecule co-factors are referred to herein as “co-enzymes.” Co-enzymes can be further divided into two groups: prosthetic molecules (e.g., co-enzymes that tightly bind, or even covalently bond to a protein) and co-substrates (e.g., co-enzymes that transiently bind to proteins).

As used herein, the term “effective amount” refers to a quantity of a composition/agent/product sufficient to achieve a desired beneficial and/or prophylactic effect, e.g., an amount that prevents, inhibits, ameliorates, cleanses, or delays the onset of an undesired result, or condition, or the physiological signs or symptoms of the condition (e.g., an adverse event associated with consumption of an alcoholic irritant). In the context of beneficial or prophylactic applications, in some embodiments, the amount of a composition/agent/product administered to the subject will depend on the type and severity of the undesired condition and on the characteristics of the individual, such as general health, age, sex, body weight and tolerance to drugs. In some embodiments, it will also depend on the degree, severity and type of condition. The skilled artisan will be able to determine appropriate dosages depending on these and other factors.

As used herein, the term “enzyme” refers to molecules or molecule aggregates that are responsible for catalyzing chemical and biological reactions. Such molecules are typically proteins, but can also comprise short peptides, RNAs, ribozymes, antibodies, and other molecules. A molecule that catalyzes chemical and biological reactions is referred to as having “enzyme activity” or having “catalytic activity.”

As used herein, in connection with the use of methods of the disclosure, the term “immediately” refers to about a period of time between steps of a method. For example, an enzyme known to enzymatically breakdown at least one alcoholic irritant can be introduced to a subject “immediately” before or “immediately” after the consumption of an alcoholic irritant (e.g., an alcoholic beverage) by the subject. In some embodiments, “immediately” refers to about 30 minutes or less, about 25 minutes or less, about 20 minutes or less, about 15 minutes or less, about 10 minutes or less, about 5 minutes or less, or about 1 minute or less.

As used herein, the term “composition” refers to the combination of an agent (e.g., an enzyme known to enzymatically breakdown at least one alcoholic irritant) with a carrier, inert or active, making the composition especially suitable for use in vivo. By the same token, dietary supplements, foods and cosmetic compositions are readily contemplated.

As used herein, a “polynucleotide” or “nucleic acid” in its broadest sense, refer to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” or “polynucleotide” refers to an oligonucleotide chain comprising individual nucleic acid residues. In some embodiments, a “nucleic acid” or “polynucleotide” is or comprises RNA; in some embodiments, a “nucleic acid” or “polynucleotide” is or comprises DNA. In some embodiments, a nucleic acid or polynucleotide is, comprises, or consists of one or more natural nucleic acid residues. In some embodiments, a nucleic acid or polynucleotide is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid or polynucleotide has one or more phosphorothioate and/or 5′-N-phosphoramidite linkages rather than phosphodiester bonds. In some embodiments, a nucleic acid or polynucleotide is, comprises, or consists of one or more natural nucleosides (e.g., adenosine, thymidine, guanosine, cytidine, uridine, deoxyadenosine, deoxythymidine, deoxy guanosine, and deoxy cytidine). In some embodiments, a nucleic acid or polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereol). In some embodiments, a nucleic acid or polynucleotide comprises one or more modified sugars (e.g., 2′-fluororibose, ribose, 2′-deoxyribose, arabinose, and hexose) as compared with those in natural nucleic acids. In some embodiments, a nucleic acid or polynucleotide has a nucleotide sequence that encodes a functional gene product such as an RNA or protein. In some embodiments, a nucleic acid or polynucleotide includes one or more introns. In some embodiments, nucleic acids or polynucleotides are prepared by one or more of isolation from a natural source, enzymatic synthesis by polymerization based on a complementary template (in vivo or in vitro), reproduction in a recombinant cell or system, and chemical synthesis. In some embodiments, a nucleic acid or polynucleotide is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid or polynucleotide is partly or wholly single stranded; in some embodiments, a nucleic acid or polynucleotide is partly or wholly double stranded. In some embodiments a nucleic acid or polynucleotide has a nucleotide sequence comprising at least one element that encodes, or is the complement of a sequence that encodes, a polypeptide. In some embodiments, a nucleic acid or polynucleotide has enzymatic activity. In some embodiments, a nucleic acid or polynucleotide is conjugated to a detectable marker (e.g., a fluorophore). Those skilled in the art, reading the present specification, will appreciate that ligation oligonucleotide sets, activating nucleic acids, and/or guide RNAs can each be engineered and/or manipulated, e.g., to incorporate nucleotide analogs, etc.

As used herein, “prevention” or “preventing” of an undesired condition, or detrimental outcome refers to results that, in a statistical sample, exhibit a reduction in the occurrence of the condition or detrimental outcome in a sample or subject administered an agent or agents relative to a control sample or subject (e.g., prevention of, for example, nausea, associated with consumption of alcoholic irritants). Such prevention is sometimes referred to as a prophylactic treatment.

As used herein, the terms “polypeptide”, “peptide”, and “protein” are used interchangeably to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full-length proteins or fragments thereof, wherein the amino acid residues are linked by covalent peptide bonds.

As used herein in connection with the use of methods of the disclosure, the term “shortly” refers to about a period of time between steps of a method. For example, an enzyme known to enzymatically breakdown at least one alcoholic irritant can be introduced to a subject “shortly” before or “shortly” after the consumption of an alcoholic irritant (e.g., an alcoholic beverage) by the subject. In some embodiments, “shortly” refers to about 35 minutes, about 45 minutes, about 1 hour, about 2 hours, about 3 hours, about 4 hours, about 6 hours, about 8 hours, about 10 hours, about 12 hours, about 16 hours, about 18 hours, or about 24 hours.

As used herein, the term “reference standard” refers to a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the term “simultaneous” use refers to the administration of at least two active ingredients (e.g., agents) by the same route and at the same time or at substantially the same time.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

As used herein, the phrase “beneficial agent, dietary supplement, food, or cosmetic agent,” as the case might be, refers to an agent that, when introduced to a subject, has an effect and/or elicits a desired biological, nutritive, physiological, cosmetic and/or pharmacological effect. In some embodiments, a beneficial agent is any substance that can be used to alleviate, ameliorate, relieve, inhibit, prevent, delay onset of, reduce severity of, and/or reduce incidence of one or more symptoms or features of an undesired condition (e.g., consumption of or exposure to one or more alcoholic irritants).

As used herein in the context of molecules, e.g., polynucleotides, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a polynucleotide may have a characteristic sequence element comprised of a plurality of nucleotide residues having designated positions relative to one another in linear or three-dimensional space. In some embodiments, a variant polypeptide or polynucleotide may differ from a reference polypeptide or polynucleotide as a result of one or more differences in amino acid or nucleotide sequence and/or one or more differences in chemical moieties (e.g., carbohydrates, lipids, phosphate groups) that are covalently components of the polypeptide or polynucleotide (e.g., that are attached to the polypeptide or polynucleotide backbone). In some embodiments, a variant polypeptide or polynucleotide shows an overall sequence identity with a reference polypeptide or polynucleotide that is at least 85%, 86%, 87%, 88%, 89%, 90%, 91%, 92%, 93%, 94%, 95%, 96%, 97%, or 99%. In some embodiments, a variant polypeptide or polynucleotide does not share at least one characteristic sequence element with a reference polypeptide or polynucleotide. In some embodiments, a reference polypeptide or polynucleotide has one or more biological activities. In some embodiments, a variant polypeptide or polynucleotide shares one or more of the biological activities of the reference polypeptide or polynucleotide. In some embodiments, a variant polypeptide or polynucleotide lacks one or more of the biological activities of the reference polypeptide or polynucleotide. In some embodiments, a variant polypeptide or polynucleotide shows a reduced level of one or more biological activities as compared to the reference polypeptide or polynucleotide. In some embodiments, a polypeptide or polynucleotide of interest is considered to be a “variant” of a reference polypeptide or polynucleotide if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or polynucleotide comprises about 10, about 9, about 8, about 7, about 6, about 5, about 4, about 3, about 2, or about 1 substituted residues as compared to a reference. Often, a variant polypeptide or polynucleotide comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or polynucleotide comprises not more than about 5, about 4, about 3, about 2, or about 1 addition or deletion, and, in some embodiments, comprises no additions or deletions, as compared to the reference. In some embodiments, a variant polypeptide or polynucleotide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or polynucleotide is one found in nature. In some embodiments, a reference polypeptide or polynucleotide is a human polypeptide or polynucleotide.

### Compositions, Routes of Administration, and Dosing

The methods, uses, and compositions of the present disclosure introduce (“administer”) a composition (e.g., an effective amount of a composition) to cleanse at least a portion of a lung of a subject (e.g., from at least one alcoholic irritant in vivo). A composition can be a solid or a liquid, including but not limited to, mists, aerosols, vapors, drops, and powders. Such compositions can be, for example, snortable, inhalable, and/or vapable.

In some embodiments, a composition comprises at least one enzyme known to enzymatically breakdown at least one alcoholic irritant and/or a polynucleotide(s) encoding the same, that can be deposited in the lung tissue of a subject to cleanse at least one alcoholic irritant that is capable of diffusing between lung tissue and the circulatory system of the subject present in the subject. The deposition of such an enzyme(s) and/or polynucleotide(s) in lung tissue of the subject can enhance the degradative efficacy of the subject, can result in a reduced level of the at least one alcoholic irritant in the subject (e.g., in the blood of the subject), and/or can, in some embodiments, result in relief from the symptoms (e.g., adverse effects) caused by an alcoholic irritant in the subject.

A composition, as used herein, is a composition comprising an agent and a carrier, inert or active, making the composition especially suitable for use in vivo. A composition (e.g., pharmaceutical, cosmetic, food, or dietary supplement) can include, for example, at least one enzyme known to enzymatically breakdown at least one alcoholic irritant. A composition can include, for example, a polynucleotide (or polynucleotides) encoding at least one enzyme known to enzymatically breakdown at least one alcoholic irritant. In some embodiments, a composition can include at least one enzyme known to enzymatically breakdown at least one alcoholic irritant, and a polynucleotide(s) encoding such enzymes. In some preferred embodiments, a composition is especially suitable for the delivery and deposition of an agent (e.g., an enzyme or a polynucleotide encoding the same) to a pulmonary tissue in a subject. In some preferred embodiments, a composition is especially suitable for the delivery and deposition of at least one enzyme known to enzymatically breakdown at least one alcoholic irritant to lung tissue of a subject.

A composition in accordance with the present disclosure may include an enzyme known to enzymatically breakdown at least one alcoholic irritant. Non-limiting examples of enzymes known to enzymatically breakdown at least one alcoholic irritant include for example, EC 1.1 enzymes (e.g., EC 1.1.1 enzymes), EC 1.2 enzymes (e.g., EC 1.2.1 enzymes), monooxygenases (EC 1.14.14.1), chloroperoxidases (EC 1.11.1.10), and laccases (EC 1.10.3.2). The enzyme can be an isolated enzyme. The enzyme can be a human enzyme (e.g., an isolated human enzyme). The enzyme can be a non-human enzyme (e.g., an isolated non-human enzyme). The enzyme can be a recombinant enzyme. The enzyme can be a wildtype enzyme or a variant thereof. In some embodiments, a variant enzyme comprises an amino acid sequence having at least 65%, at least 70%, at least 75%, at least 80%, at least 85%, at least 90%, at least 95%, or at least 99% sequence identity to a corresponding wild-type enzyme.

EC 1.1 enzymes act on the CH—OH group of donors and comprises dehydrogenases that act on primary alcohols, secondary alcohols, and hemi-acetals. The EC 1.1.1 subclass of EC 1.1 enzymes utilize NAD or NADP as cofactors, and include, for example, alcohol dehydrogenases (ADH). In some embodiments, a composition comprises an EC 1.1 enzyme or a variant thereof and/or a polynucleotide encoding the same. In some embodiments, a composition comprises an EC 1.1.1 enzyme or a variant thereof and/or a polynucleotide encoding the same. In some embodiments, a composition comprises an EC 1.1.1.1 enzyme or a variant thereof and/or a polynucleotide encoding the same. In some embodiments, a composition comprises an EC 1.1.1.2 enzyme or a variant thereof and/or a polynucleotide encoding the same. In some embodiments, a composition comprises an alcohol dehydrogenase (ADH) or a variant thereof and/or a polynucleotide encoding the same.

EC 1.2 enzymes act on the aldehyde or oxo group of donors and comprises enzymes that oxidize aldehydes to the corresponding acids. Oxo groups may be oxidized either with addition of water and cleavage of a carbon-carbon bond or, in the case of ring compounds, by addition of the elements of water and dehydrogenation. The EC 1.2.1 subclass of EC 1.2 enzymes utilize NAD⁺ or NADPas cofactors, and include, for example, acetaldehyde dehydrogenase (EC 1.2.1.10). In some embodiments, a composition comprises an EC 1.2 enzyme or a variant thereof and/or a polynucleotide encoding the same. In some embodiments, a composition comprises an EC 1.2.1 enzyme or a variant thereof and/or a polynucleotide encoding the same. In some embodiments, a composition comprises an EC 1.2.1.10 enzyme or a variant thereof and/or a polynucleotide encoding the same. In some embodiments, a composition comprises an acetaldehyde dehydrogenase (ALDH) or a variant thereof and/or a polynucleotide encoding the same.

In some embodiments, a composition comprises a category EC 1.1 enzyme, a category EC 1.2 enzyme, a variant thereof, or a combination thereof and/or a polynucleotide(s) encoding the same. In some embodiments, a composition comprises one or more of alcohol dehydrogenase (ADH) or a variant thereof, aldehyde dehydrogenase (ALDH) or a variant thereof, or combinations thereof and/or a polynucleotide(s) encoding the same. In some embodiments, a composition comprises a human alcohol dehydrogenase (e.g., NP_000662.3, NP_000664.3, NP_001159976.1, NP_001273579.1, NP_000659.2, NP_000658.1, NP_000663.1, NP_001095940.1, NP_000660.1). In some embodiments, a composition comprises a human aldehyde dehydrogenase (e.g., NP_001191818.1, NP_000681.2, NP 000680.2, NP_001356077.1, NP_001356075.1, NP_001356068.1, NP_001356067.1, NP 001356066.1, NP_001356065.1, NP_001026976.1, NP_000373.1, NP_000683.3, NP 001317079.1, NP 001128640.1, NP_001128639.1, NP_000682.3). In some embodiments, a composition further comprises an NADH oxidase (e.g., WP_011667127.1, WP 020089694.1, WP 011668263.1, WP_003637851.1, NP_001369451.1, NP_001369445.1, NP_001369446.1, NP_001369449.1).

Monooxygenase are enzymes that catalyze the incorporation of one atom of molecular oxygen into a substrate, with the other being reduced to water. In some embodiments, a composition comprises a monooxygenase or a variant thereof and/or a polynucleotide encoding the same.

Chloroperoxidases are enzymes that catalyze the chlorination of organic compounds. In some embodiments, a composition comprises a chloroperoxidase or a variant thereof and/or a polynucleotide encoding the same.

Laccases are multicopper oxidases. In some embodiments, a composition comprises a laccase or a variant thereof and/or a polynucleotide encoding the same.

In some embodiments, a composition can comprise one or more co-factors, co-enzymes, and/or co-substrates. In some embodiments, a composition comprises at least one enzyme known to enzymatically breakdown at least one alcoholic irritant and/or a polynucleotide(s) encoding the same, and further comprises one or more co-factors, co-enzymes, co-substrates, or combinations thereof. Examples of co-factors include, without limitation, nicotinamide adenine dinucleotide (NAD⁺), nicotinamide adenine dinucleotide phosphate (NADP⁺), flavin adenine dinucleotide (FAD), and pyrroloquinoline quinone (PQQ). Selection of the appropriate co-factor for use in accordance with an enzyme known to enzymatically breakdown at least one alcoholic irritant is well within the level of one of ordinary skill in the art. In some such embodiments, compositions can comprise one or more of nicotinamide adenine dinucleotide (NAD⁺), nicotinamide adenine dinucleotide phosphate (NADP⁺), flavin adenine dinucleotide (FAD), and pyrroloquinoline quinone (PQQ).

In some embodiments, a composition comprises an alcohol dehydrogenase (or a variant thereof) and NAD and/or NADP. In some embodiments, a composition comprises an acetaldehyde dehydrogenase (or a variant thereof) and NAD and/or NADP⁺. In some embodiments, a composition comprises an alcohol dehydrogenase (or a variant thereof), an acetaldehyde dehydrogenase (or a variant thereof) and NAD and/or NADP.

In some embodiments, a composition comprises a polynucleotide encoding an alcohol dehydrogenase (or a variant thereof) and NAD and/or NADP⁺. In some embodiments, a composition comprises a polynucleotide encoding an acetaldehyde dehydrogenase (or a variant thereof) and NAD and/or NADP⁺. In some embodiments, a composition comprises a polynucleotide encoding an alcohol dehydrogenase (or a variant thereof) and an acetaldehyde dehydrogenase (or a variant thereof) and NAD and/or NADP⁺. In some embodiments, a composition comprises a polynucleotide encoding an alcohol dehydrogenase (or a variant thereof), a polynucleotide encoding an acetaldehyde dehydrogenase (or a variant thereof) and NAD and/or NADP⁺.

Without wishing to be bound by any one theory, it is understood that the relative amount of reduced to oxidized co-factor can play an important role for the equilibrium of an enzymatic reaction. Thus, regeneration of co-factors is important for degradative efficiency. In some embodiments, compositions of the present disclosure comprise an enzyme for co-factor regeneration or a polynucleotide encoding the same. In some embodiments, a composition comprises NADH oxidase or a variant thereof (an enzyme which catalyzes the oxidation of NADH to yield NAD and H₂O (“water-forming NADH oxidase”), H₂O₂ (“hydrogen peroxide forming NADH oxidase”) or both) or a polynucleotide encoding the same. In some embodiments, a composition comprises at least one enzyme known to enzymatically break down at least one alcoholic irritant (or a variant thereof) and further comprises an enzyme for co-factor regeneration (e.g., NADH oxidase or a variant thereof) or a polynucleotide encoding the same. In some embodiments, a composition comprises a polynucleotide(s) encoding at least one enzyme known to enzymatically break down at least one alcoholic irritant (or a variant thereof) and further comprises an enzyme for co-factor regeneration (e.g., NADH oxidase or a variant thereof) (or a polynucleotide encoding the same).

In some embodiments, a composition comprises an alcohol dehydrogenase (or a variant thereof), NADH oxidase or a variant thereof (or a polynucleotide encoding the same), NAD⁺ and/or NADP⁺. In some embodiments, a composition comprises an acetaldehyde dehydrogenase (or a variant thereof), NADH oxidase or a variant thereof (or a polynucleotide encoding the same), and NAD and/or NADP. In some embodiments, a composition comprises an alcohol dehydrogenase (or a variant thereof), an acetaldehyde dehydrogenase (or a variant thereof), and a NADH oxidase or a variant thereof (or a polynucleotide encoding the same). In some embodiments, a composition comprises an alcohol dehydrogenase (or a variant thereof), an acetaldehyde dehydrogenase (or a variant thereof), NADH oxidase or a variant thereof (or a polynucleotide encoding the same), and NAD and/or NADP⁺.

In some embodiments, a composition comprises a polynucleotide encoding an alcohol dehydrogenase (or a variant thereof), NADH oxidase or a variant thereof (or a polynucleotide encoding the same), and NAD and/or NADP. In some embodiments, a composition comprises a polynucleotide encoding an acetaldehyde dehydrogenase (or a variant thereof), NADH oxidase or a variant thereof (or a polynucleotide encoding the same), and NAD and/or NADP. In some embodiments, a composition comprises a polynucleotide encoding an alcohol dehydrogenase (or a variant thereof), an acetaldehyde dehydrogenase (or a variant thereof), and NADH oxidase or a variant thereof (or a polynucleotide encoding the same). In some embodiments, a composition comprises a polynucleotide encoding an alcohol dehydrogenase (or a variant thereof) and an acetaldehyde dehydrogenase (or a variant thereof), NADH oxidase or a variant thereof (or a polynucleotide encoding the same), and NAD and/or NADP. In some embodiments, a composition comprises a polynucleotide encoding an alcohol dehydrogenase (or a variant thereof), a polynucleotide encoding an acetaldehyde dehydrogenase (or a variant thereof), NADH oxidase or a variant thereof (or a polynucleotide encoding the same), and NAD and/or NADP.

In some embodiments, wherein a hydrogen-peroxide forming NADH oxidase is utilized, catalase (EC 1.11.1.6) can be used to convert hydrogen peroxide to oxygen and water. Thus, in some embodiments, a composition comprises a catalase or a variant thereof and/or a polynucleotide encoding a catalase. In some embodiments, a catalase or a variant thereof and/or a polynucleotide encoding a catalase may be present in any composition described herein comprising NADH-oxidase or a variant thereof (and/or polynucleotide encoding the same), wherein the NADH oxidase is a hydrogen peroxide forming NADH oxidase.

A cosmetic is generally considered a composition or formulation specifically prepared for administration to a subject to address condition (e.g., the presence of an alcoholic irritant in a subject).

In some embodiments, the agent(s) (e.g., an enzyme known to enzymatically breakdown at least one alcoholic irritant or a polynucleotide encoding such an enzyme) can be formulated with little or no excipient or carrier. In some embodiments, the agent(s) can be formulated such that the majority of the formulation is excipient or carrier. In brief, one of skill in the art will tailor the formulation to have a suitable amount of excipient or carrier based on the needs/condition of the subject, the kind and extent of the condition of the subject; the properties of the agent or agents to be delivered and the selected mode of introduction of the particular agent or agents.

In certain embodiments, a composition may comprise at least one agent other than the at least one enzyme known to enzymatically breakdown at least one alcoholic irritant (or a polynucleotide encoding the same). In certain embodiments, a composition may further comprise at least one agent that increases enzyme residence time in the lungs in addition to the at least one enzyme known to enzymatically breakdown at least one alcoholic irritant (or a polynucleotide encoding the same). Agents that may increase enzyme residence time in the lungs include, for example and without limitation, liposomes, cyclodextrins, and low molecular weight amino acids.

Compositions may contain an effective amount of one or more of the agent or agents as described herein and may optionally be disbursed (e.g. dissolved, suspended or otherwise) in an physiologically acceptable carrier. The components of the composition(s) may also be capable of being commingled with other agents or active agents, and with each other, in a manner such that there is no interaction that would substantially impair the desired efficacy.

As stated above, an “effective amount” refers to any amount of a particular agent that is sufficient to achieve a desired biological effect. Combined with the teachings provided herein, by choosing among the various agent(s) and weighing factors such as potency, relative bioavailability, subject body weight, severity of adverse side-effects and mode of administration, an effective prophylactic (i.e. preventative) or beneficial introduction regimen can be planned which does not cause substantial unwanted toxicity and yet is effective to address the particular condition of a particular subject in a beneficial way. The effective amount of an agent for any particular indication can vary depending on such factors as the condition of the subject, the particular agent(s) being administered, the size of the subject, the age of the subject, the overall health of the subject and/or the severity of the condition. The effective amount may be determined during pre-clinical trials and/or clinical trials by methods familiar to physicians and clinicians. One of ordinary skill in the art can empirically determine the effective amount of a particular agent or agents without necessitating undue experimentation. A maximum dose may be used, that is, the highest safe dose according to some medical judgment. Multiple doses per day may be contemplated to achieve appropriate levels of compounds/agents. Appropriate levels can be determined by, for example, measurement of the patient's peak or sustained plasma level of the drug. “Dose” and “dosage” are used interchangeably herein. A dose may be administered by oneself, by another or by way of a device (e.g., an inhaler).

For any composition described herein, the effective amount can, for example, be initially determined from animal models. An effective dose can also be determined from human data for compounds/agents which have been tested in humans and for compounds/agents which are known to exhibit similar pharmacological activities, such as other related active agents. The applied dose can be adjusted based on the relative bioavailability and potency of the administered compound/agent. Adjusting the dose to achieve maximal efficacy based on the methods described above and other methods as are well-known in the art is well within the capabilities of the ordinarily skilled artisan.

Agents (alone or as formulated in a composition/cosmetic) for use in methods described herein can be tested in suitable animal model systems. Suitable animal model systems include, but are not limited to, rats, mice, chicken, cows, monkeys, rabbits, pigs, minipigs and the like, prior to testing in human subjects. In vivo testing of any animal model system known in the art can be used prior to administration to human subjects. In some embodiments, dosing can be tested directly in humans.

Dosage, toxicity and efficacy of any agents or compositions (e.g., formulations or cosmetics comprising compositions described herein), other/additional agents, or mixtures thereof can be determined by standard procedures in cell cultures or experimental animals, e.g., for determining the LD50 (the dose lethal to 50% of the population) and the ED50 (the dose effective in 50% of the population). The dose ratio between toxic and beneficial effects is the “therapeutic” index and it can be expressed as the ratio LD50/ED50. Agents that exhibit high “therapeutic” indices are advantageous.

An exemplary introduction regimen can, for example, entail administration once per day, twice per day, thrice per day, once a week, or once a month. In beneficial applications, a relatively high dosage at relatively short intervals is sometimes required until progression of the condition (e.g., the level of alcoholic irritant in the subject, such as the level in the blood of the subject) is delayed, reduced, or terminated, or until the subject shows partial or complete amelioration of symptoms of a condition. In some embodiments, a single administration is sufficient to delay, reduce, terminate, or ameliorate symptoms of a condition.

For use, an effective amount of the agent (alone or as formulated) can be introduced to a subject by any mode that delivers the composition to the desired surface (e.g., lung tissue). Introducing a composition may be accomplished by any suitable route but under conditions that do not support a significant or substantial permeation of the agent beyond pulmonary tissue (i.e., minimization of systemic introduction). Routes of introduction include, for example, inhalation, intratracheally, topically, and intranasally. Preferably, introduction (“administration”) is not carried out intraperitoneally, intradermally, ophthalmically, intrathecally, intracerebroventricularly, iontophoretically, transmucosally, intravitreally, or intramuscularly . . . . In some embodiments, an agent (e.g., an enzyme known to enzymatically breakdown one or more alcoholic irritants) or a composition comprising the same resides substantially in the subject's pulmonary tissue following introduction of the enzyme, variant thereof, or combination thereof. In some embodiments, an agent (e.g., an enzyme known to enzymatically breakdown one or more alcoholic irritants) or a composition comprising the same is introduced under conditions that inhibit or do not support systemic delivery of said agent or composition. In some embodiments, an agent (e.g., an enzyme known to enzymatically breakdown one or more alcoholic irritants) or a composition comprising the same is introduced to the at least a portion of the lung of said subject in a manner that minimizes a systemic introduction into said subject. In some embodiments, an agent is introduced in a manner that does not include pulmonary transmucosal delivery of the agent to said subject, which results in an undesired systemic delivery. In some preferred embodiments, delivery (“introduction”) of a composition is accomplished by inhalable administration. Administration includes self-administration, administration by another, and administration by a device (e.g., e.g., a vaper, a dry powder inhaler or vapor pump, but preferably not an infusion pump). An agent disclosed herein (e.g., enzymes known to enzymatically breakdown at least one alcoholic irritant, polynucleotides encoding the same) can be delivered to the subject in a formulation or cosmetic (i.e., a composition). Formulations and cosmetics can be prepared by, for example, dissolving or suspending an agent disclosed herein (e.g., enzymes, polypeptides, polynucleotides encoding the same, and combinations thereof) in water, a solvent, a physiologically acceptable carrier, salt, (e.g., NaCl or sodium phosphate), buffering agents, preservatives, compatible carriers, adjuvants, and optionally other physiologically acceptable ingredients.

The compositions (e.g. a formulation or cosmetic) can include a carrier (e.g., a physiologically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars (e.g., trehalose), polyalcohols such as mannitol, sorbitol, or sodium chloride in the composition.

One may dilute or increase the volume of the agent(s) or composition(s) with an inert material. These diluents could include carbohydrates, especially mannitol, lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

An anti-frictional agent may be included in the formulation of the agent(s) or composition(s) to prevent sticking during the formulation process. Lubricants may be used as a layer between the agent and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the agent during formulation and to aid rearrangement during compression might be added. The glidants may include starch, talc, pyrogenic silica and hydrated silicoaluminate.

To aid dissolution of the agent(s) or composition(s) into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation or cosmetic as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation or cosmetic disclosed herein or derivative either alone or as a mixture in different ratios.

For topical administration, the agent(s) or composition(s) may be formulated as solutions, gels, ointments, creams, suspensions, etc. as are well-known in the art. Solutions, gels, ointments, creams or suspensions may be administered topically.

For administration (introduction) by inhalation, agent(s) (e.g., enzymes known to enzymatically breakdown at least one alcoholic irritant) or composition(s) for use according to the present application may be conveniently delivered in the form of an aerosol, a mist, a vapor, or a drop presentation from, for example, by pressurized packs, a nebulizer, and/or vaporizers (e.g., vape pens), optionally with the use of a suitable propellant, e.g., dichlorodifluoromethane, trichlorofluoromethane, dichlorotetrafluoroethane, carbon dioxide or other suitable gas or liquid, such as propylene glycol. In some embodiments, the formulation, cosmetic and/or agent(s) can be delivered in the form of an aerosol spray from a pressurized container or dispenser, in the form of a vapor from a vaporizer (e.g., vape pen), which contains a suitable propellant, e.g., a gas such as carbon dioxide, a liquid such has propylene glycol, or in the form of a mist from a nebulizer. For example, certain methods include those described in U.S. Pat. No. 6,468,798. In the case of a pressurized aerosol, the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the composition/agent and a suitable powder base such as lactose or starch. Alternatively, the agent(s) or composition(s) may be in powder form for constitution with a suitable vehicle, e.g., sterile pyrogen-free water or a suitable buffer, before use.

For administration (introduction) by inhalation, agent(s) (e.g., enzymes known to enzymatically breakdown at least one alcoholic irritant) or composition(s) for use according to the present application may formulated for dry powder inhalation. Dry powder inhalation of macromolecules (e.g., enzymes of the present disclosure) can utilize a formulation comprising poly(ethylene glycol)-co-poly(glycerol-adipate-co-@-pentadecalactone) as a biodegradable, polymer carrier, as described in Tawfeek H M, et al. (Int J Pharm. 2013 Jan. 30; 441 (1-2): 611-9; incorporated by reference herein in its entirety).

Intranasal delivery of an agent(s) or composition(s) is also contemplated, such as snorting. Preferably, the intranasal delivery does not allow the passage of agent(s) or composition(s) to the blood stream after administering the agent(s) or composition(s) to the nose. In some embodiments, less than about 50%, less than about 40%, less than about 30%, less than about 20%, or less than about 10% of the agent introduced enters the systemic circulation after intranasal introduction (delivery). Formulations for intranasal delivery include, for example, those with dextran or cyclodextran.

For intranasal administration, one type of useful device is a small, hard bottle to which a metered dose sprayer is attached. In some embodiments, the metered dose is delivered by drawing a composition (in solution form) into a chamber of defined volume, which chamber has an aperture dimensioned to aerosolize and aerosol formulation by forming a spray when a liquid in the chamber is compressed. The chamber is compressed to administer the compound(s)/agent(s) or composition(s). In a specific embodiment, the chamber is a piston arrangement. Such devices are commercially available.

Alternatively, a plastic squeeze bottle with an aperture or opening dimensioned to aerosolize an aerosol formulation by forming a spray when squeezed can be used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the agent(s) or composition(s).

Compositions can also be or comprise a mist which can be inhaled or administered (introduced) by, for example, intranasal delivery. Compositions can also be or comprise a drop which can be administered (introduced) by, for example, intranasal delivery.

Contemplated for use in the practice of this technology are a wide range of mechanical devices designed for inhalable delivery of agents or compositions, including but not limited to nebulizers, metered dose inhalers, powder inhalers, a dry powder or vapor pump (but not an infusion pump) and vaporizers (e.g., vape pens), all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the agent(s) or composition(s). Typically, each formulation is specific to the type of device employed and may involve the use of an appropriate propellant material, in addition to the usual diluents, adjuvants and/or carriers useful in beneficial introduction regimens. Also, the use of liposomes, microcapsules, microspheres, nanoparticles, nanospheres, inclusion complexes, or other types of carriers is contemplated.

Formulations suitable for use with a nebulizer, either jet or ultrasonic, can, for example, comprise agent(s) or composition(s) dissolved in water at a concentration of about 0.01 to 50 mg of biologically active composition per mL of solution. The formulation may also include a buffer and optionally a simple sugar (e.g., for stabilization and regulation of osmotic pressure). The nebulizer formulation may also contain a surfactant, to reduce or prevent surface induced aggregation of the compound(s)/agent(s) or composition(s) disclosed herein caused by atomization of the solution in forming the aerosol.

Formulations for use with a metered-dose inhaler device may generally comprise a finely divided powder comprising the agent(s) or composition(s) disclosed herein suspended in a propellant with the aid of a surfactant. The propellant may be any conventional material employed for this purpose, such as a chlorofluorocarbon, a hydrochlorofluorocarbon, a a hydrofluorocarbon, or hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device may comprise a finely divided dry powder containing the agent(s) or composition(s) and may also include a bulking agent, such as lactose, sorbitol, sucrose, or mannitol in amounts which facilitate dispersal of the powder from the device, e.g., 50 to 90% by weight of the formulation. The agent(s)/composition(s) can advantageously be prepared in particulate or nanoparticulate form with an average particle size of less than 10 micrometers (μm), most preferably 0.5 to 5 μm, for most effective delivery to the deep lung.

Formulations for use with a vaporizer (e.g., a vape pen) may generally comprise a liquid comprising the agent(s) or composition(s) disclosed herein and a propellant and optionally, concentrated flavors. The propellant may be any conventional material employed for this purpose, such as propylene glycol and/or glycerol.

### Formulations and Cosmetics

Any of the formulations (which can also be referred to as a composition, or cosmetic when formulated for introduction to a subject) described in the section above entitled: “Compositions, Routes of Administration, and Dosing” can be applied to produce a composition (i.e. a formulation or cosmetic) suitable for administration to a subject in need thereof. Thus, in some embodiments, this application is directed to compositions, formulations, and cosmetics suitable for introduction (“administration”) to a subject who has consumed an alcoholic irritant or otherwise has an increased, systemic level of an alcoholic irritant.

In some embodiments, a composition, formulation, or cosmetic is administered by intratracheally, topically, intranasally or by inhalation so long as such introduction is under conditions that do not support a significant or substantial permeation of the composition, formulation, or cosmetic beyond pulmonary tissue (i.e., minimization of systemic introduction) For example, in some embodiments, less than about 50%, less than about 40%, less than about 30%, less than about 20%, or less than about 10% of the composition, formulation, or cosmetic introduced permeates mucosal tissue (i.e., introduction is not transmucosal) into a subject's blood or serum.

### Methods and In Vivo Alcohol Irritant Degradation

The present disclosure provides, among other things, methods of cleansing at least a portion of a lung of a subject and methods of freshening the breath of a subject. In some embodiments, a method of cleansing at least a portion of a lung of a subject comprises introducing to at least a portion of a lung of a subject a composition comprising at least one enzyme known to enzymatically breakdown at least one alcoholic irritant present systemically in said subject, including at least a portion of said lung. In some embodiments, a method of freshening the breath of a subject comprises introducing to at least a portion of a lung of a subject a composition comprising at least one enzyme known to enzymatically breakdown at least one alcoholic irritant present systemically in said subject, including at least a portion of said lung.

In some embodiments, a method of cleansing at least a portion of a lung of a subject comprises allowing an enzymatic breakdown of at least a portion of any alcoholic irritant that has diffused or migrated from the subject's circulatory system into the subject's lung or lung mucous.

In some embodiments, a method of freshening the breath of a subject comprises allowing an enzymatic breakdown of at least a portion of any alcoholic irritant that has diffused or migrated from the subject's circulatory system into the subject's lung or lung mucous.

As defined herein above, alcoholic irritants refer to an identifiable, isolatable substance comprising at least one hydroxyl group (—OH) that is freely circulating within a subject via a circulatory system and capable of diffusing between lung tissue and the circulatory system of a subject. Alcoholic irritants can include, for example, primary alcohols (e.g., ethanol, benzylic alcohol, phenylethanol, geraniol and retinol) and secondary alcohols (e.g., 2-propanol, 2-butanol). Alcoholic irritants can also include, for example, straight- or branched-chain lower aliphatic alcohols (e.g., methanol, ethanol, propanol, isopropyl alcohol and the like). Further examples of alcoholic irritants include, without limitation, diethylene glycol and ethylene glycol.

Alcoholic irritants are typically water-soluble compounds and once consumed (e.g., by humans), move into water spaces throughout the body, including the systemic circulation. It is understood that alcoholic irritants can be transported from the systemic circulation to the pulmonary circulation where they can readily diffuse from the pulmonary blood supply into the lungs. Alcoholic irritants can then be degraded in the lungs or can return to the systemic circulation. Without wishing to be bound by any one theory, it is understood that introduction of compositions comprising at least one enzyme known to enzymatically breakdown at least one alcoholic irritant to at least a portion of a lung of a subject using the methods described herein can facilitate in vivo degradation of alcoholic irritants, effectively converting the lungs to a tunable cleansing site, with cleansing capacities introduced by exogenously added enzymes. Such cleansing capabilities are understood to also reduce the level of an alcoholic irritant (e.g., ethanol) in the breath, thereby freshening the breath of a subject.

In some embodiments, the at least one alcoholic irritant comprises a primary alcohol. In some embodiments, the at least one alcoholic irritant comprises a secondary alcohol. In some embodiments, the at least one alcoholic irritant comprises a straight- and/or branch-chain lower aliphatic alcohol. In some embodiments, the at least one alcoholic irritant is selected from the group consisting of methanol, ethanol, propanol, isopropyl alcohol, benzylic alcohol, phenylethanol, geraniol, retinol, and ethylene glycol, and combinations thereof. In some embodiments, the at least one alcoholic irritant comprises methanol or ethanol. In some embodiments, the at least one alcoholic irritant comprises methanol. In some embodiments, the at least one alcoholic irritant comprises methanol or ethanol. In some embodiments, the at least one alcoholic irritant comprises methanol or ethanol. In some embodiments, the at least one alcoholic irritant comprises propanol. In some embodiments, the at least one alcoholic irritant comprises isopropyl alcohol. In some embodiments, the at least one alcoholic irritant comprises benzylic alcohol. In some embodiments, the at least one alcoholic irritant comprises benzylic alcohol. In some embodiments, the at least one alcoholic irritant comprises phenylethanol. In some embodiments, the at least one alcoholic irritant comprises geraniol. In some embodiments, the at least one alcoholic irritant comprises retinol. In some embodiments, the at least one alcoholic irritant comprises ethylene glycol.

The introducing (administering) step of methods described herein can be accomplished by any means that results in contacting pulmonary tissue of the subject with either the at least one enzyme known to enzymatically breakdown at least one alcoholic irritant, a polynucleotide(s) encoding the at least one enzyme known to enzymatically breakdown at least one alcoholic irritant, or both the at least one enzyme known to enzymatically breakdown at least one alcoholic irritant and the polynucleotide(s) encoding the at least one enzyme known to enzymatically breakdown at least one alcoholic irritant (e.g., wherein the conditions do not support a significant or substantial permeation of the agent beyond pulmonary tissue (i.e., minimization of systemic introduction)). In some embodiments, the introducing step is accomplished by transducing a plurality of cells present in pulmonary tissue to express said at least one enzyme known to enzymatically breakdown at least one alcoholic irritant. In some embodiments, the plurality of cells is transduced with DNA or a construct thereof. In some embodiments, the plurality of cells is transduced with mRNA or a construct thereof. The DNA, mRNA, or constructs thereof may be formulated with or without a physiologically acceptable carrier. In some embodiments, less than about 50%, less than about 40%, less than about 30%, less than about 20%, or less than about 10% of the composition introduced permeates mucosal tissue (i.e., introduction is not transmucosal) into a subject's blood or serum after introduction.

In some embodiments, the composition comprising the at least one enzyme known to breakdown at least one alcoholic irritant (and/or a polynucleotide(s) encoding the same) is introduced before, during, or after the consumption of the at least one alcoholic irritant by said subject. In some embodiments, the composition comprising the at least one enzyme known to breakdown at least one alcoholic irritant (and/or a polynucleotide(s) encoding the same) is introduced immediately before, during, or immediately after the consumption of the at least one alcoholic irritant by said subject. In some embodiments, the composition comprising the at least one enzyme known to breakdown at least one alcoholic irritant (and/or a polynucleotide(s) encoding the same) is introduced shortly before, during, or shortly after the consumption of the at least one alcoholic irritant by said subject.

Alcoholic irritants can be consumed by a plurality of methods. For example, alcoholic irritants can be consumed by one or more of snorting, inhaling, sublingual absorption, enema, vaginal absorption, eyeballing, injection, and ingestion.

In some embodiments, an alcoholic irritant is comprised in a solid or liquid preparation. Such preparations can comprise, for example and without limitation, a pill, a gummy, a sublingual, a lozenge, an injectable, a food, a beverage, a household product, or combinations thereof.

In some embodiments, methods of the present disclosure comprising cleansing (e.g., cleansing from an alcoholic irritant, such as method, or ethanol) at least a portion of a lung of a subject, wherein said subject is contemplating a consumption of, is in a process of consuming, or has consumed one or more solid or liquid preparations comprising an alcoholic irritant (e.g., an alcoholic beverage, a household product). In some embodiments, methods of the present disclosure comprising cleansing (e.g., cleansing from an alcoholic irritant, such as method, or ethanol) at least a portion of a lung of a subject, wherein said subject consumed one or more alcoholic beverages.

In some embodiments, methods of the present disclosure comprise freshening the breath of a subject, wherein said subject is contemplating a consumption of, is in a process of consuming, or has consumed one or more solid or liquid preparations comprising an alcoholic irritant (e.g., an alcoholic beverage, a household product). In some embodiments, methods of the present disclosure freshening the breath of a subject, wherein said subject consumed one or more alcoholic beverages.

In some embodiments, the at least one alcoholic irritant is consumed in a household product, such as cleaning agents, personal care (e.g., mouthwash, cough syrup) and topical products, and pesticides. In some embodiments, a household product is snorted. In some embodiments, a household product is inhaled (e.g., using a vaporizer, such as a vape pen). In some embodiments, a household product is consumed by sublingual absorption. In some embodiments, a household product is consumed by enema. In some embodiments, a household product is consumed by vaginal absorption. In some embodiments, a household product is consumed by eyeballing. In some embodiments, a household product is consumed by injection (e.g., intravenously) into a subject. In some embodiments, a household product is consumed by ingestion. In some embodiments, a household product is consumed by huffing.

In some embodiments, the at least one alcoholic irritant is consumed in an alcoholic beverage (e.g., liquor, beer, wine). In some embodiments, an alcoholic beverage is snorted. In some embodiments, an alcoholic beverage is inhaled (e.g., using a vaporizer, such as a vape pen). In some embodiments, an alcoholic beverage is consumed by sublingual absorption. In some embodiments, an alcoholic beverage is consumed by enema. In some embodiments, an alcoholic beverage is consumed by vaginal absorption. In some embodiments, an alcoholic beverage is consumed by eyeballing. In some embodiments, an alcoholic beverage is consumed by injection (e.g., intravenously) into a subject. In some embodiments, an alcoholic beverage or food comprising alcohol (e.g., alcoholic gummy bears, popsicles) is consumed by ingestion.

An enzyme known to enzymatically breakdown at least one alcoholic irritant for use in accordance with technologies of the present disclosure can include any enzyme known to enzymatically breakdown at least one alcoholic irritant. Such enzymes include, for example, EC 1.1 enzymes (e.g., EC 1.1.1 enzymes), EC 1.2 enzymes (e.g., EC 1.2.1 enzymes), monooxygenases, chloroperoxidases, and laccases. In some embodiments, said enzyme a variant thereof, or a combination thereof resides substantially in the subject's pulmonary tissue following introduction of the enzyme, variant thereof, or combination thereof. In some embodiments, said enzyme, a variant thereof, or a combination thereof is introduced under conditions that inhibit or do not support systemic delivery of said enzyme, a variant thereof, or a combination thereof to said subject. In some embodiments, said enzyme, a variant thereof, or a combination thereof is introduced to the at least a portion of the lung of said subject in a manner that minimizes a systemic introduction into said subject. In some embodiments, said enzyme is introduced in a manner that does not include pulmonary transmucosal delivery of the agent to said subject, which results in an undesired systemic delivery. In some embodiments, said enzyme, a variant thereof, or a combination thereof excludes an introduction of an ADH/KRED bound to at least one long-acting molecule or complexing molecule. In still other embodiments, an introduction excludes an introduction via inhalation of an atomized solution of an ADH/KRED bound to at least one long-acting molecule or complexing molecule.

In some embodiments, an enzyme known to enzymatically breakdown at least one alcoholic irritant for use in accordance with technologies of the present disclosure is PEGylated. Without wishing to be bound by any one theory, it is understood that enzyme PEGylation can reduce transmucosal transport of such enzymes (e.g., increasing the fraction of enzyme known to enzymatically breakdown at least one alcoholic irritant resident in the subject's pulmonary tissue and/or minimizing a systemic introduction of said enzyme into said subject).

EC 1.1 enzymes act on the CH—OH group of donors and comprises dehydrogenases that act on primary alcohols, secondary alcohols, and hemi-acetals. The EC 1.1.1 subclass of EC 1.1 enzymes utilize NAD or NADP as cofactors, and include, for example, alcohol dehydrogenases. In some embodiments, the at least one enzyme known to enzymatically breakdown at least one alcoholic irritant for use in accordance with methods of the present disclosure is or comprises an EC 1.1 enzyme or a variant thereof. In some embodiments, the at least one enzyme known to enzymatically breakdown at least one alcoholic irritant for use in accordance with methods of the present disclosure is or comprises an EC 1.1.1 enzyme or a variant thereof. In some embodiments, the at least one enzyme known to enzymatically breakdown at least one alcoholic irritant for use in accordance with methods of the present disclosure is or comprises an EC 1.1.1.1 enzyme or a variant thereof. In some embodiments, the at least one enzyme known to enzymatically breakdown at least one alcoholic irritant for use in accordance with methods of the present disclosure is or comprises an EC 1.1.1.2 enzyme or a variant thereof. In some embodiments, the at least one enzyme known to enzymatically breakdown at least one alcoholic irritant for use in accordance with methods of the present disclosure is or comprises an alcohol dehydrogenase (ADH) or variant thereof.

EC 1.2 enzymes act on the aldehyde or oxo group of donors and comprises enzymes that oxidize aldehydes to the corresponding acids. Oxo groups may be oxidized either with addition of water and cleavage of a carbon-carbon bond or, in the case of ring compounds, by addition of the elements of water and dehydrogenation. The EC 1.2.1 subclass of EC 1.2 enzymes utilize NAD or NADP as cofactors, and include, for example, acetaldehyde dehydrogenase (EC 1.2.1.10). In some embodiments, the at least one enzyme known to enzymatically breakdown at least one alcoholic irritant for use in accordance with methods of the present disclosure is or comprises an EC 1.2 enzyme or a variant thereof. In some embodiments, the at least one enzyme known to enzymatically breakdown at least one alcoholic irritant for use in accordance with methods of the present disclosure is or comprises an EC 1.2.1 enzyme or a variant thereof. In some embodiments, the at least one enzyme known to enzymatically breakdown at least one alcoholic irritant for use in accordance with methods of the present disclosure is or comprises an EC 1.2.1.10 enzyme or a variant thereof. In some embodiments, the at least one enzyme known to enzymatically breakdown at least one alcoholic irritant for use in accordance with methods of the present disclosure is or comprises an acetaldehyde dehydrogenase (ALDH) or variant thereof.

In some embodiments, the at least one enzyme known to enzymatically breakdown at least one alcoholic irritant comprises an EC 1.1 enzyme or a variant thereof, an EC 1.2 enzyme or a variant thereof, or a combination thereof.

In some embodiments, the at least one enzyme known to enzymatically breakdown at least one alcoholic irritant for use in accordance with methods of the present disclosure is or comprises a monooxygenase.

In some embodiments, the at least one enzyme known to enzymatically breakdown at least one alcoholic irritant for use in accordance with methods of the present disclosure is or comprises a chloroperoxidase.

In some embodiments, the at least one enzyme known to enzymatically breakdown at least one alcoholic irritant for use in accordance with methods of the present disclosure is or comprises a laccase.

In some embodiments, a composition comprising at least one enzyme known to enzymatically breakdown at least one alcoholic irritant comprises a composition, formulation, or cosmetic as described elsewhere herein.

In some embodiments, the present disclosure provides methods of cleansing ethanol from at least a portion of a lung of a subject comprising introducing to at least a portion of a lung of a subject a composition comprising alcohol dehydrogenase (ADH), a variant thereof, or a combination thereof.

In some embodiments, the present disclosure provides methods of cleansing ethanol from at least a portion of a lung of a subject comprising introducing to at least a portion of a lung of a subject a composition comprising aldehyde dehydrogenase (ALDH), a variant thereof, or a combination thereof.

In some embodiments, the present disclosure provides methods of cleansing ethanol from at least a portion of a lung of a subject comprising introducing to at least a portion of a lung of a subject a composition comprising alcohol dehydrogenase (ADH), aldehyde dehydrogenase (ALDH), variants thereof, or combinations thereof.

In some embodiments, the present disclosure provides methods of cleansing an alcoholic irritant from at least a portion of a lung of a subject comprising: (a) introducing to at least a portion of a lung of a subject an effective amount of a composition comprising alcohol dehydrogenase (ADH), aldehyde dehydrogenase (ALDH), human variants thereof, or combinations thereof; and (b) allowing an enzymatic breakdown of at least a portion of any alcoholic irritant that has diffused or migrated from the subject's circulatory system into the subject's lung and/or lung mucous.

In some embodiments, the present disclosure provides methods of freshening the breath of a subject comprising introducing to at least a portion of a lung of a subject a composition comprising alcohol dehydrogenase (ADH), a variant thereof, or a combination thereof.

In some embodiments, the present disclosure provides methods of freshening the breath of a subject comprising introducing to at least a portion of a lung of a subject a composition comprising aldehyde dehydrogenase (ALDH), a variant thereof, or a combination thereof.

In some embodiments, the present disclosure provides methods of freshening the breath of a subject comprising introducing to at least a portion of a lung of a subject a composition comprising alcohol dehydrogenase (ADH), aldehyde dehydrogenase (ALDH), variants thereof, or combinations thereof.

In some embodiments, the present disclosure provides methods of freshening the breath of a subject comprising: (a) introducing to at least a portion of a lung of a subject an effective amount of a composition comprising alcohol dehydrogenase (ADH), aldehyde dehydrogenase (ALDH), human variants thereof, or combinations thereof; and (b) allowing an enzymatic breakdown of at least a portion of any alcoholic irritant that has diffused or migrated from the subject's circulatory system into the subject's lung and/or lung mucous.

In some embodiments, enzymes known to enzymatically breakdown at least one alcoholic irritant require the presence of co-factors (e.g., co-enzymes, e.g., co-substrates). Examples of co-factors include, without limitation, nicotinamide adenine dinucleotide (NAD⁺), nicotinamide adenine dinucleotide phosphate (NADP⁺), flavin adenine dinucleotide (FAD), pyrroloquinoline quinone (PQQ)). Selection of the appropriate co-factor for use in accordance with an enzyme known to enzymatically breakdown at least one alcoholic irritant is well within the level of one of ordinary skill in the art.

In some embodiments, compositions of the present disclosure comprises cofactors (e.g., co-enzymes, co-substrates) as described elsewhere herein. In some embodiments, compositions comprising at least one enzyme known to enzymatically break down at least one alcoholic irritant further comprises coenzymes, cofactors, co-substrates, or combinations thereof. In some such embodiments, compositions can comprise one or more of nicotinamide adenine dinucleotide (NAD⁺), nicotinamide adenine dinucleotide phosphate (NADP⁺), flavin adenine dinucleotide (FAD), pyrroloquinoline quinone (PQQ).

Without wishing to be bound by any one theory, it is understood that the relative amount of reduced to oxidized cofactor can play an important role for the equilibrium of a reaction. Thus, regeneration of co-factors is important for degradative efficiency. In some embodiments, methods of the present disclosure further comprise introducing an additional enzyme for co-factor regeneration. In some such embodiments, methods of the present disclosure further comprise introducing NADH oxidase (or a variant thereof), an enzyme which catalyzes the oxidation of NADH to yield NAD and H₂O (“water-forming NADH oxidase”), H₂O₂ (“hydrogen peroxide-forming NADH oxidase”) or both. In some embodiments, a composition comprises NADH oxidase or a variant thereof, as described elsewhere herein. In some embodiments, a composition comprising at least one enzyme known to enzymatically break down at least one alcoholic irritant further comprises NADH oxidase or a variant thereof. In some embodiments, wherein methods of the present disclosure further comprise introducing a NADH oxidase or a variant thereof to a subject, and wherein the NADH oxidase or variant thereof is a hydrogen peroxidase forming NADH oxidase, such methods further comprises introducing a catalase.

In some embodiments, methods of the present disclosure are utilized to cleanse at least a portion of a lung of a subject and/or freshen the breath of a subject from an alcoholic irritant selected from the group consisting of methanol, ethanol, propanol, isopropyl alcohol, benzylic alcohol, phenylethanol, geraniol, retinol, and ethylene glycol, and combinations thereof. In some embodiments, methods of the present disclosure are utilized to cleanse at least a portion of a lung of a subject and/or freshen the breath of a subject from an alcoholic irritant, wherein the alcoholic irritant is methanol or ethanol.

Methods of the present disclosure can improve the rate of cleansing of an alcoholic irritant (e.g., ethanol) from a subject's blood (e.g., a human subject's blood) per minute compared to a control subject (e.g., a subject wherein no enzyme known to enzymatically breakdown the at least one alcoholic irritant was introduced. In some embodiments, the rate of cleansing is improved by at least about 1%, 2%, 3%, 4%, 5%, 6%, 7%, 8%, 9%, or 10% compared to a control subject. In some embodiments, rate of cleansing the at least one alcoholic irritant from the subject's blood per minute is improved by at least about 1%, 2%, 3%, 4%, 5%, 6%, 7%, 8%, 9%, or 10% compared to a control subject wherein no enzyme known to enzymatically breakdown the least one alcoholic irritant was introduced. In some embodiments, rate of cleansing ethanol from the subject's blood per minute is improved by at least about 1%, 2%, 3%, 4%, 5%, 6%, 7%, 8%, 9%, or 10% compared to a control subject wherein no ADH, ALDH, variants thereof, or combinations thereof were introduced. In some embodiments, rate of cleansing the alcoholic irritant from the subject's blood per minute is improved by at least about 1%, 2%, 3%, 4%, 5%, 6%, 7%, 8%, 9%, or 10% compared to a control subject wherein no ADH, ALDH, variants thereof, or combinations thereof were introduced. In some embodiments, rate of cleansing the alcoholic irritant from the subject's blood per minute is improved by at least about 1%, 2%, 3%, 4%, 5%, 6%, 7%, 8%, 9%, or 10% compared to a control subject wherein no ADH, ALDH, NADH oxidase, variants thereof, or combinations thereof were introduced. In some embodiments, rate of cleansing ethanol from the subject's blood per minute is improved by at least about 1%, 2%, 3%, 4%, 5%, 6%, 7%, 8%, 9%, or 10% compared to a control subject wherein no ALDH, variants thereof, or combinations thereof were introduced.

Methods of the present disclosure can reduce the level of an alcoholic irritant (e.g., ethanol) in the blood, breath, and/or other biological samples from the subject (e.g., urine, saliva) of the subject. For example, the level of an alcoholic irritant in the blood of the subject can be reduced by about 10% or more, about 15% or more, about 20% or more, about 25% or more about 30% or more, about 40% or more, about 50% or more, about 60% or more, about 75% or more, about 90% or more relative to the level present before application of the presently disclosed technologies. In some embodiments, the level of an alcoholic irritant in the blood, breath, and/or other biological samples from the subject (e.g., urine, saliva) of the subject can be reduced by about 10% or more, about 15% or more, about 20% or more, about 25% or more about 30% or more, about 40% or more, about 50% or more, about 60% or more, about 75% or more, about 90% or more about one hour, about two hours, about four hours, about 6 hours, about 10 hours, about 12 hours, about 18 hours, about 24 hours, about 36 hours, or about 48 hours after introducing a composition comprising at least one enzyme known to enzymatically breakdown at least one alcoholic irritant to at least a portion of a lung of the subject relative to the level present before application of the presently disclosed technologies. In some embodiments, the level of an alcoholic irritant in the blood, breath, and/or other biological samples from the subject (e.g., urine, saliva) of the subject can be reduced by about 10% or more, about 25% or more, about 50% or more, about 75% or more, about 90% or more about one hour after introducing a composition comprising at least one enzyme known to enzymatically breakdown at least one alcoholic irritant to at least a portion of a lung of the subject relative to the level present before application of the presently disclosed technologies.

In some embodiments, the level of an alcoholic irritant in the blood, breath, and/or other biological samples from the subject (e.g., urine, saliva) of the subject can be reduced by about 10% or more, about 15% or more, about 20% or more, about 25% or more about 30% or more, about 40% or more, about 50% or more, about 60% or more, about 75% or more, about 90% or more relative to an appropriate reference standard. In some embodiments, the level of an alcoholic irritant in the blood, breath, and/or other biological samples from the subject (e.g., urine, saliva) of the subject can be reduced by about 10% or more, about 15% or more, about 20% or more, about 25% or more about 30% or more, about 40% or more, about 50% or more, about 60% or more, about 75% or more, about 90% or more about one hour, about two hours, about four hours, about 6 hours, about 10 hours, about 12 hours, about 18 hours, about 24 hours, about 36 hours, or about 48 hours after introducing a composition comprising at least one enzyme known to enzymatically breakdown at least one alcoholic irritant to at least a portion of a lung of the subject relative to an appropriate reference standard. In some embodiments, the level of an alcoholic irritant in the blood, breath, and/or other biological samples from the subject (e.g., urine, saliva) of the subject can be reduced by about 10% or more, about 25% or more, about 50% or more, about 75% or more, about 90% or more about one hour after introducing a composition comprising at least one enzyme known to enzymatically breakdown at least one alcoholic irritant to at least a portion of a lung of the subject relative to an appropriate reference standard.

Technologies to measure alcohol irritant levels in subject are readily known in the art and their use in accordance with the present disclosure is well within the level of one of ordinary skill in the art. For example, alcohol irritant levels can be measured by administering a breathalyzer test, a saliva test, a urine test, and/or a blood test which can utilize, for example, infrared spectrophotometry or gas chromatography (e.g., headspace gas chromatography). See, e.g., Jones, A W. Alcohol, its analysis in blood and breath for forensic purposes, impairment effects, and acute toxicity. WIREs Forensic Sci. 2019; 1: e1353, incorporated herein by reference.

### Subject Populations

In some embodiments, a subject of the present disclosure (e.g., who undergoes methods of cleansing described herein) includes, but is not limited to, humans and non-human vertebrates. In some embodiments, a subject in accordance with the methods of the disclosure comprise, for example, a mammal. In some such embodiments, a mammal includes, for example and without limitation, a household pet (e.g., a dog, a cat, a rabbit, a ferret, a hamster, etc.), a livestock or farm animal (e.g., a cow, a pig, a sheep, a goat, a pig, a chicken or another poultry), a horse (e.g., a thoroughbred horse), a monkey, a laboratory animal (e.g., a mouse, a rat, a rabbit, etc.), and the like. In a preferred embodiment, a subject of the present disclosure is a human. In some embodiments, technologies of the present disclosure can be practiced in any subject that is likely to benefit from cleansing at least a portion of a lung of a subject (e.g., from an alcoholic irritant).

In some embodiments, methods of the present disclosure can be practiced in a subject that has consumed one or more alcoholic irritants (e.g., one or more alcoholic beverages, a household product). In some embodiments, methods of the present disclosure can be practiced in a subject contemplating a consumption of, is in a process of consuming, or has consumed one or more solid or liquid preparations comprising an alcohol (e.g., an alcoholic beverage).

In some embodiments, a subject of the present disclosure is a human newborn (e.g., birth to 1 month of age), a human infant (e.g., 1 month to 1 year of age), a human child (1 year to 12 years of age), a human adolescent (13 years to 17 years of age), or a human adult (18 years of age or older).

### EXAMPLES

These examples are provided for illustrative purposes only and not to limit the scope of the claims provided herein.

### Example 1: Reduction of Circulating Ethanol Levels by ADH Delivery to Mouse Lung

The present Example demonstrates the effect of alcohol dehydrogenase (ADH) enzyme delivery to the lungs of mice on ethanol levels in circulation after acute ethanol intoxication.

### Materials and Methods

Enzyme and Enzyme Solution: Alcohol dehydrogenase (ADH) from Saccharomyces cerevisiae lyophilized powder), ≥300 units/mg protein, was utilized (Sigma-Aldrich; A7011). NAD⁺ utilized was obtained commercially (Sigma-Aldrich; SKU 10127965001). Enzyme solution was prepared by diluting 100 mg of ADH in 2 mL of phosphate buffered saline (PBS) on the day the study protocol began. The Enzyme plus NAD⁺ solution was prepared by diluting 100 mg of ADH and 100 mg of NAD⁺ in 2 mL of PBS.

Ethanol Solution: Non-denatured ethanol was diluted to a 20% weight/volume solution with PBS (4.3M ethanol solution).

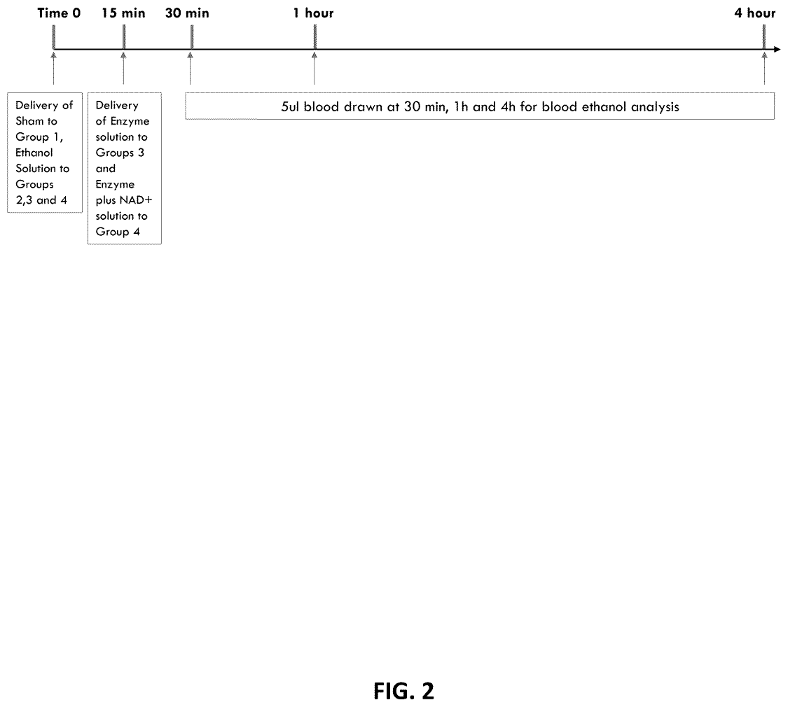

Blood Draw Dilution: Blood was drawn from mice at time points zero, 30 minutes, 1 hour and/or 4 hours after ethanol gavage (FIG. 2) and diluted 100× in buffer. The blood was processed for plasma, 50 μl of blood from the tail vein at each time point was collected using 125 μl heparinized capillary tube (Fisher Sci Cat #14-915-64). Using a pipette the heparinized blood was forced out by air of the capillary tube and into a 0.6 ml mct. The tubes were kept on ice and subsequently spun down to recover the plasma. Plasma was removed and placed into a new tube and kept on ice until needed for the same day assay. 1-5 μl of plasma was removed for the assay and any residual plasma was placed at −80° C. in labeled box.

Ethanol Detection: Ethanol was detected using the Sigma-Aldrich Ethanol Assay Kit (MAK076) according to the manufacturer's protocol for colorimetric detection. Several dilutions for a 5 μL blood draw were tested to ensure the sample was within the linear range of the standard curve. Working in a lab space exposed to ethanol vapors was avoided.

### Results

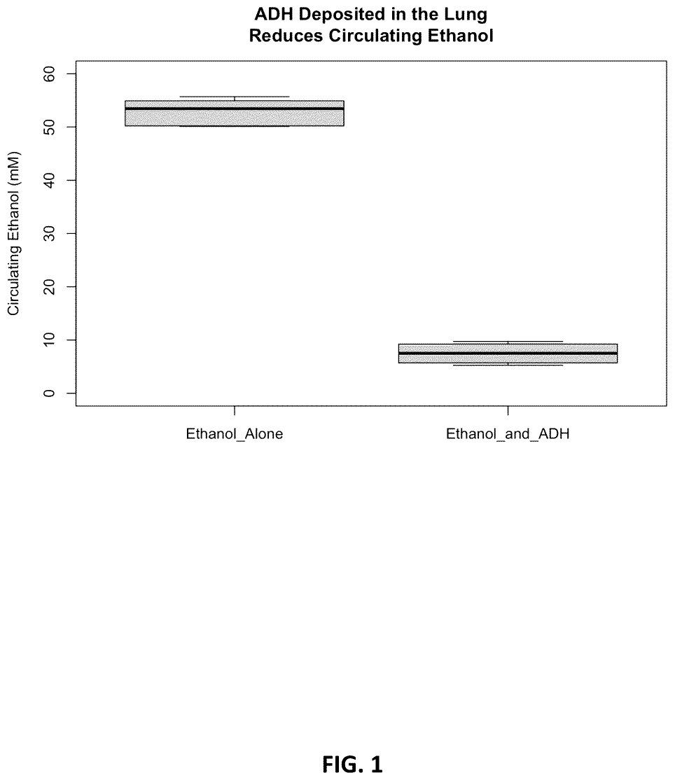

To evaluate the effect of ADH enzyme delivery to the lungs of a mouse on ethanol levels in circulation after acute ethanol intoxication, mice were administered ethanol by oral gavage alone, or in combination with intratracheal administration of ADH. As shown in FIG. 1, circulating ethanol levels were reduced in mice treated with pulmonary delivered ADH relative to control.

To further evaluate the effect of ADH enzyme delivery to the lungs of a mouse on ethanol levels in circulation after acute ethanol intoxication, control mice administered a sham 2 g/kg gavage with PBS (300 μl) delivered at time zero. Ethanol control and experimental group mice were administered ethanol 2 g/kg (300 μl of 20% ethanol solution for a 25 g mouse) by oral gavage at time zero. Experimental group mice were subsequently administered either (i) ADH (1 mg enzyme); or (ii) both ADH and co-factor, NAD⁺ (2 mg ADH, 0.15 mmole NAD⁺) by intratracheally delivery to lungs 15 minutes after ethanol gavage. Blood was drawn (5 μl) from mice of each control and experimental group at time zero, 30 minutes, 1 hour and/or 4 hours after ethanol gavage. A schematic outlining the study protocol is shown in FIG. 2.

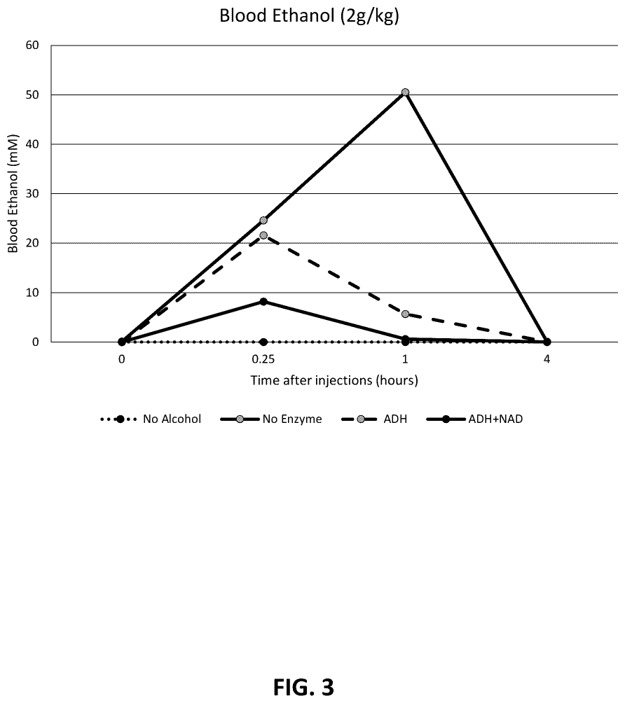

As shown in FIG. 3, blood alcohol levels were reduced in mice treated with pulmonary delivered ADH relative to animals that were not administered ADH. These results suggest that the pulmonary deposition of ADH enzyme is sufficient to reduce ethanol levels in the blood of a subject.

An additional study evaluating the effect of ADH enzyme delivery to the lungs of a mouse on ethanol levels in circulation after acute ethanol intoxication, ethanol control mice were administered 4 g/kg at time one. Experimental group mice were administered 4 g/kg at time one and ADH and NAD″ (2 mg ADH; 0.15 mmol NAD) at time 3 after blood draw. Experimental time points are summarized below:
T1: blood draw prior to ethanol administration.
T2: blood draw at 50 minutes post-ethanol administration.
T3: blood draw at 60 minutes post-ethanol administration, before intratracheal administration of ADH for the experimental group.
T4: blood draw at 10 minutes post-ADH delivery.
T5: blood draw at 20 minutes post-ADH delivery.
T6: blood draw at 30 minutes post-ADH delivery.
T7: blood draw at 40 minutes post-ADH delivery.
T8: blood draw at 50 minutes post-ADH delivery.
T9: blood draw at 60 minutes post-ADH delivery.
T10: blood draw at 70 minutes post-ADH delivery.
T11: blood draw at 80 minutes post-ADH delivery.
T12: blood draw at 90 minutes post-ADH delivery.

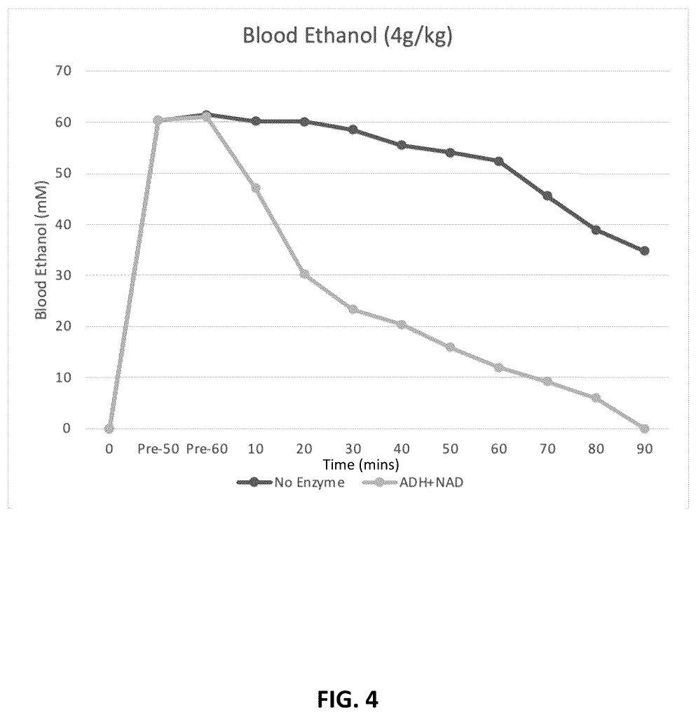

Results in FIG. 4 demonstrate alcohol clearance in the blood is accelerated in mice by pulmonary administration of ADH and NAD⁺ (triangles) after acute ethanol intoxication compared to control (crosses).

### Example 2: Alcohol Clearance in Mice by Pulmonary Delivery of an Ethanol Metabolizing Enzyme

The present example demonstrates the effect of alcohol dehydrogenase (ADH) enzyme delivery to the lungs of mice on ethanol levels in circulation after acute ethanol intoxication utilizing the same protocol described in Example 1. However, the Abcam Ethanol Detection kit was utilized for ethanol detection and the experimental group delivered ADH and NAD⁺ were administered 1 mg ADH, 0.15 mmole NAD⁺ (as opposed to 2 mg ADH, 0.15 mmole NAD⁺ utilized in Example 1).

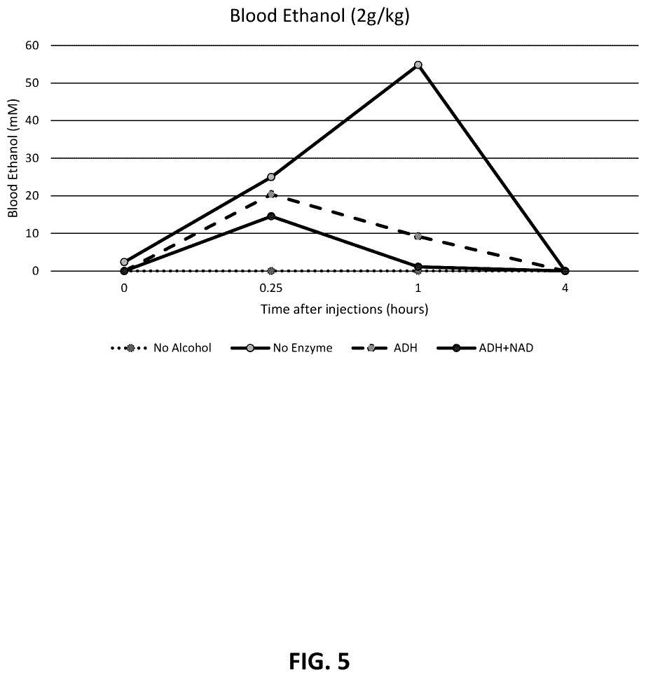

Results demonstrated that blood alcohol levels were reduced in mice pulmonary delivered ADH relative to animals that were not administered ADH, further supporting that the pulmonary deposition of ADH enzyme is sufficient to reduce ethanol levels in the blood of a subject (FIG. 5).

### Example 3: Reduction of Circulating Ethanol Levels by ADH and NADH Oxidase Delivery to Mouse Lung

The present Example describes the expected effect of one or more of alcohol dehydrogenase (ADH), NADH oxidase, and catalase delivery to the lungs of mice on ethanol levels in circulation after acute ethanol intoxication.

### Materials and Methods

Methods of blood draw dilution and ethanol detection can be completed as is described in Example 1.

Enzyme and Enzyme Solution: Alcohol dehydrogenase from Saccharomyces cerevisiae lyophilized powder), ≥300 units/mg protein, is utilized (Sigma-Aldrich A7011). NAD⁺ is obtained commercially (Sigma-Aldrich SKU 10127965001). ADH enzyme solution is prepared by diluting 100 mg of ADH in 2 mL of phosphate buffered saline (PBS) on the day the study protocol begins. The Enzyme plus NAD⁺ solution is prepared by diluting 100 mg of enzyme and 100 mg of NAD⁺ in 2 mL of PBS. NADH-oxidase (water forming; NOWF) and NADH-oxidase (peroxide forming, NOPF) are obtained commercially. NADH oxidase solutions are prepared. Catalase is obtained commercially and catalase solution is prepared.

Ethanol Solution: Non-denatured ethanol is diluted to a 70% volume/volume solution.

### Results

To evaluate the expected effect of one or more of ADH, NADH oxidase, and catalase delivery to the lungs of mice on ethanol levels in circulation after acute ethanol intoxication, at time zero, a blood sample is drawn prior to ethanol administration. Mice are then administered 4 g/kg ethanol (273 μl of 70% ethanol solution for a 25 g mouse) at time zero by oral gavage. An additional blood sample is drawn 50 minutes after ethanol oral gavage and at 60 minutes after ethanol oral gavage (prior to enzyme introduction).

Some NADH oxidases are “water-forming” in which the co-product is water (NOWF), while others are hydrogen peroxide forming (NOPF). In the latter case, catalase can be used to convert peroxide to oxygen and water.

Experimental groups can include mice administered either (i) ADH and NAD⁺ (2 mg enzyme or 600 U, 0.15 mmole NAD⁺) 60 minutes after ethanol oral gavage; (ii) ADH, NAD⁺, and NOWF enzyme (2 mg ADH or 600 U, 0.15 mmole NAD⁺; 10 mg of enzyme of 300 U NOWF) 60 minutes after ethanol oral gavage; (iii) NOWF enzyme 60 minutes after ethanol oral gavage; and (iv) ADH, NAD⁺, NOPF and Catalase (2 mg ADH, 0.15 mmole NAD⁺; 0.4 mg or 15 U NOPF; 0.15 mg or >300 U catalase) 60 minutes after oral gavage.

Additional blood samples are drawn from the mice about every 10 minutes after enzyme delivery for about 90 minutes.

It is expected that introduction of one or more of NADH oxidase and catalase with ADH and NAD⁺ by pulmonary delivery to mice will promote reduction of circulating ethanol levels compared to introduction of ADH and NAD⁺ alone.

### Example 4: In Vivo Reduction of Circulating Ethanol Levels is Enhanced by Pulmonary Delivery of ADH Relative to Systemic Delivery of ADH

To evaluate whether the in vivo reduction of circulating ethanol in mice is enhanced when ADH is delivered to the lungs as opposed to systemically, 4 kg/g of ethanol is administered to mice by oral gavage. One hour after oral gavage of ethanol, a group of mice are administered either (i) 1 mg ADH intravenously; or (ii) 1 mg ADH intratracheally. Administration of an aqueous biological inert solution comprising no ADH is administered to a group of mice one hour after oral gavage of ethanol as a negative control.

It is expected blood alcohol concentration increases during the hour preceding ADH administration and significantly decreases during the about 40 minutes following ADH deposition in the lung. It is expected that no decrease in blood alcohol concentration, or a smaller decrease in blood alcohol concentration compared to mice intratracheally administered ADH, is observed for mice intravenously administered ADH. Without wishing to be bound by any one theory, it is understood that oxygenation levels in the lung likely aid in ADH-mediated ethanol oxidation and may be responsible for the accelerated rate of blood alcohol clearance observed in mice treated with intratracheally administered ADH compared to intravenously administered ADH.

### Example 5: Alcohol Clearance in Mice by Pulmonary Delivery of an Ethanol Metabolizing Enzyme-Use of Commercial ALDH and ADH

The present example demonstrates the combined effect of ADH and ALDH enzyme delivery to the lungs of mice on ethanol levels in circulation after acute ethanol intoxication. The combined effect of ADH and ALDH has a stronger effect to reduce the amount of alcohol than each of the enzymes alone.

### Materials and Methods

Enzyme and Enzyme Solution: Alcohol dehydrogenase (ADH) from Saccharomyces cerevisiae lyophilized powder), ≥300 units/mg protein, was utilized (Sigma-Aldrich; A7011). NAD utilized was obtained commercially (Sigma-Aldrich; SKU 10127965001). The Enzyme plus NAD solution was prepared by diluting 100 mg of ADH and 100 mg of NAD in 2 mL of PBS. ADH plus NAD⁺ solution was prepared by diluting 100 mg of ADH plus 100 mg NAD⁺ in 2 mL of phosphate buffered saline (PBS) on the day the study protocol began.

Aldehyde dehydrogenase (ALDH) (lyophilized powder), ≥2.0 units/mg protein potassium-activated, 50 mg total (Cymit; CAS: 9028-88-0) was utilized. ALDH plus NAD⁺ solution was prepared by diluting 50 mg ALDH and 100 mg NAD⁺ in 0.500 mL of PBS on the day the study protocol began. Any unused ALDH plus NAD⁺ solution was stored in a −80° C.

Ethanol Solution: Non-denatured ethanol was diluted to a 70% volume/volume solution.

Ethanol Detection: Ethanol was detected using the Sigma-Aldrich Ethanol Assay Kit (MAK076) according to the manufacturer's protocol for colorimetric detection. Several dilutions for a 5 μL blood draw were tested to ensure the sample was within the linear range of the standard curve. Working in a lab space exposed to ethanol vapors was avoided.

### Results

To evaluate the combined effect of ADH and ALDH enzyme delivery to the lungs of mice on ethanol levels in circulation after acute ethanol intoxication, ethanol control and experimental group mice were administered 4 g/kg ethanol by oral gavage (182 μL of 70% ethanol solution for a 25 g mouse) at time one. After 60 minutes, experimental group mice were intratracheally administered either (i) ADH and NAD⁺ (2 mg or 600 U ADH; 0.15 mmol NAD⁺); (ii) ADH, NAD⁺, and ALDH (2 mg or 600 U ADH; 0.15 mmol NAD⁺; 10 mg or 20 U of ALDH); or (iii) ALDH and NAD⁺ (10 mg or 20 U ALDH; 0.15 mmol NAD⁺) at time 3. Blood was drawn every 10 minutes to measure ethanol and acetaldehyde levels. Experimental time points are summarized below:
T1: blood draw prior to ethanol administration.
T2: blood draw at 50 minutes post-ethanol administration.
T3: blood draw at 60 minutes post-ethanol administration, before intratracheal administration of enzyme/NAD⁺ for the experimental groups.
T4: blood draw at 10 minutes post-enzyme/NAD⁺ delivery.
T5: blood draw at 20 minutes post-enzyme/NAD⁺ delivery.
T6: blood draw at 30 minutes post-enzyme/NAD⁺ delivery.
T7: blood draw at 40 minutes post-enzyme/NAD⁺ delivery.
T8: blood draw at 50 minutes post-enzyme/NAD⁺ delivery.
T9: blood draw at 60 minutes post-enzyme/NAD⁺ delivery.
T10: blood draw at 70 minutes post-enzyme/NAD⁺ delivery.
T11: blood draw at 80 minutes post-enzyme/NAD⁺ delivery.
T12: blood draw at 90 minutes post-enzyme/NAD⁺ delivery.

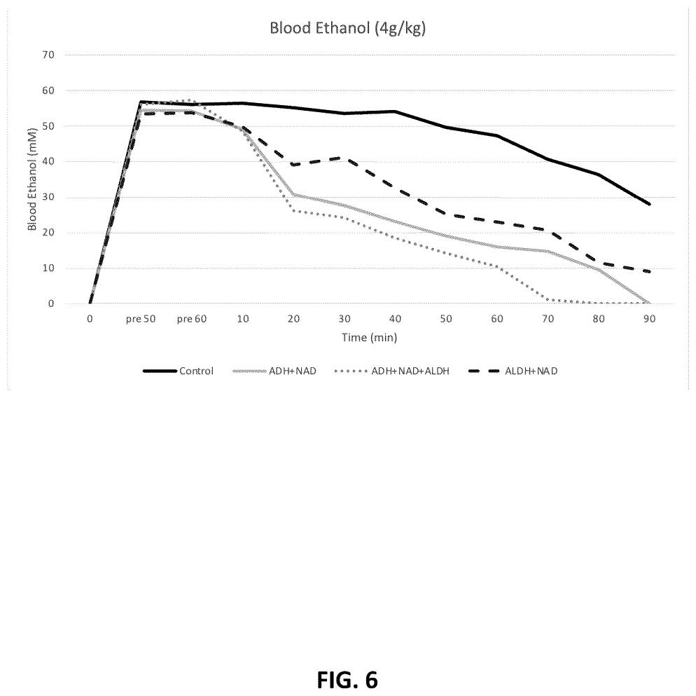

Results reported in FIG. 6 demonstrate that ADH has a stronger effect on removing alcohol compared to ALDH alone. The combined effect of ADH and ALDH has a stronger effect to reduce the amount of alcohol than each of the enzymes alone.

### Example 6: Alcohol Clearance in Mice by Pulmonary Delivery of an Ethanol Metabolizing Enzyme-Use of a Human ALDH2 Variant and Yeast ADH

The present example demonstrates the combined effect of ADH and a variant ALDH enzyme delivery to the lungs of mice on ethanol levels in circulation after acute ethanol intoxication. ADH lowered the amount of alcohol in the blood, and this effect was enhanced by the addition of ALDH to ADH, resulting in a faster degradation of alcohol.

### Materials and Methods

Enzyme and Enzyme Solution: Alcohol dehydrogenase (ADH) from Saccharomyces cerevisiae lyophilized powder), ≥300 units/mg protein, was utilized (Sigma-Aldrich; A7011). NAD⁺ utilized was obtained commercially (Sigma-Aldrich; SKU 10127965001). The Enzyme plus NAD⁺ solution was prepared by diluting 100 mg of ADH and 100 mg of NAD⁺ in 2 mL of PBS. ADH plus NAD⁺ solution was prepared by diluting 200 mg of ADH plus 200 mg NAD⁺ in 2 mL of phosphate buffered saline (PBS) on the day the study protocol began. A variant of human ALDH was utilized.

Ethanol Solution: Non-denatured ethanol was diluted to a 70% volume/volume solution.

Ethanol Detection: Ethanol was detected using the Abcam Ethanol Assay Kit (ab65343) according to the manufacturer's protocol for colorimetric detection. Several dilutions for a 5 μL blood draw were tested to ensure the sample was within the linear range of the standard curve. Working in a lab space exposed to ethanol vapors was avoided.

### Results

To evaluate the combined effect of ADH and a variant ALDH enzyme delivery to the lungs of mice on ethanol levels in circulation after acute ethanol intoxication, ethanol control and experimental group mice were administered 2 g/kg ethanol by oral gavage (90 μL of 70% ethanol solution for a 25 g mouse) at time one. After 60 minutes, experimental group mice were intratracheally administered either (i) ADH and NAD⁺ (2 mg or 600 U ADH; 0.15 mmol NAD⁺); or (ii) ADH, NAD⁺, and variant ALDH (2 mg or 600 U ADH; 0.15 mmol NAD⁺; 1.2 mg variant ALDH) at time 3. Blood was drawn every 15 minutes to measure ethanol levels. Experimental time points are summarized below:
T1: blood draw prior to ethanol administration.
T2: blood draw at 50 minutes post-ethanol administration.
T3: blood draw at 60 minutes post-ethanol administration, before intratracheal administration of enzyme/NAD⁺ for the experimental groups.
T4: blood draw at 15 minutes post-enzyme/NAD⁺ delivery.
T5: blood draw at 30 minutes post-enzyme/NAD⁺ delivery.
T6: blood draw at 45 minutes post-enzyme/NAD⁺ delivery.
T7: blood draw at 60 minutes post-enzyme/NAD⁺ delivery.
T8: blood draw at 75 minutes post-enzyme/NAD⁺ delivery.
T9: blood draw at 90 minutes post-enzyme/NAD⁺ delivery.

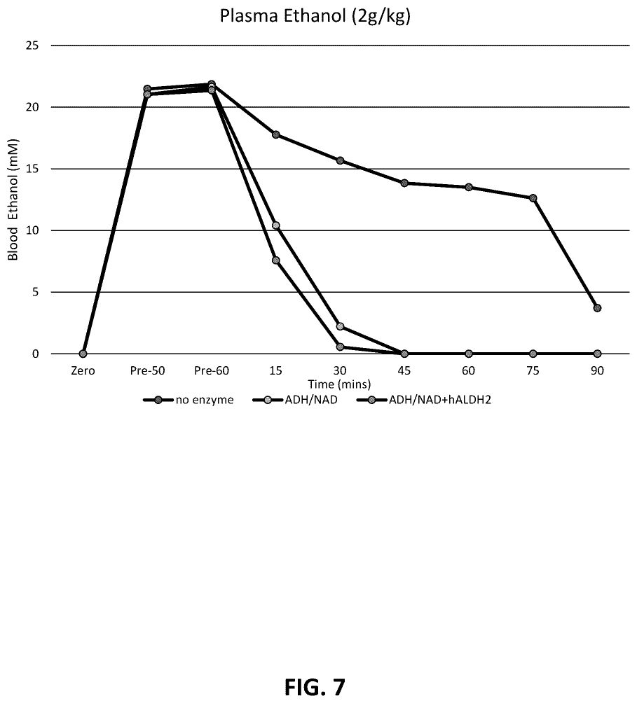

Results shown in FIG. 7 demonstrate that ADH lowered the amount of alcohol in the blood and this effect was enhanced by the addition of the variant ALDH to ADH, resulting in faster degradation of the alcohol.

### Example 7: Alcohol Clearance in Mice by Pulmonary Delivery of an Ethanol Metabolizing Enzyme—Use of a NADH Oxidase (NOX) Variant, a Human ALDH2 Variant, and Commercial Yeast ADH

The present example demonstrates that ADH lowers ethanol levels in circulation after acute ethanol intoxication in mice, and that this effect was enhanced by the additional delivery of the ALDH2 variant or a combination of the ALDH variant the NOX variant with ADH, resulting in a faster degradation of ethanol.

### Materials and Methods

Enzyme and Enzyme Solution: Alcohol dehydrogenase (ADH) from Saccharomyces cerevisiae lyophilized powder), ≥300 units/mg protein, was utilized (Sigma-Aldrich; A7011). ADH solution was prepared by diluting 200 mg of ADH in 2 mL of phosphate buffered saline (PBS) on the day the study protocol began. A variant of human ALDH (hALDH2) and a variant of the Lactobacillus brevis NOX enzyme were utilized. The variant hALDH2 and variant NOX enzymes were generated in Escherichia coli and each comprise an N-terminal HIS-tag to facilitate purification. A variant hALDH2 solution was prepared at a concentration of 60 mg/mL and a variant NOX solution was prepared at 50 mg/mL.

Ethanol Solution: Non-denatured ethanol was diluted to a 70% volume/volume solution.

Ethanol Detection: Ethanol was detected using the Sigma-Aldrich Ethanol Assay Kit (MAK076) according to the manufacturer's protocol for colorimetric detection. Several dilutions for a 5 μL blood draw were tested to ensure the sample was within the linear range of the standard curve. Working in a lab space exposed to ethanol vapors was avoided.

### Results

To evaluate the combined effect of ADH, a variant ALDH enzyme, and a variant NOX enzyme delivery to the lungs of mice on ethanol levels in circulation after acute ethanol intoxication, ethanol control and experimental group mice were administered 2 g/kg ethanol by oral gavage (90 μL of 70% ethanol solution for a 25 g mouse) at time one. After 60 minutes, experimental group mice were intratracheally administered either (i) ADH (300 μg ADH); (ii) ADH and variant ALDH (300 μg ADH; 2.52 mg variant ALDH); or (iii) ADH, variant ALDH, and variant NOX (300 μg ADH; 2.52 mg variant ALDH; 250 μg variant NOX) at time 3. Blood was drawn every 15 minutes to measure ethanol and acetaldehyde levels. Experimental time points are summarized below:
T1: blood draw prior to ethanol administration.
T2: blood draw at 50 minutes post-ethanol administration.
T3: blood draw at 60 minutes post-ethanol administration, before intratracheal administration of enzyme(s) for the experimental groups.
T4: blood draw at 15 minutes post-enzyme(s) delivery.
T5: blood draw at 30 minutes post-enzyme(s) delivery.
T6: blood draw at 45 minutes post-enzyme(s) delivery.
T7: blood draw at 60 minutes post-enzyme(s) delivery.
T8: blood draw at 75 minutes post-enzyme(s) delivery.
T9: blood draw at 90 minutes post-enzyme(s) delivery.

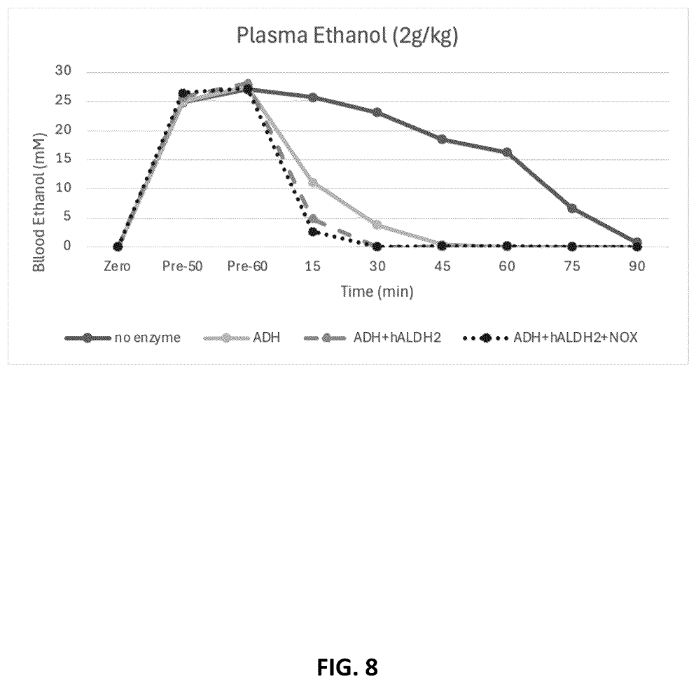

Results shown in FIG. 8 demonstrate that ADH lowered the amount of alcohol in the blood and this effect was enhanced by the addition of the variant ALDH or variant ALDH and variant NOX to ADH, resulting in faster degradation of the alcohol.

### Example 8: Alcohol Clearance in a Human Subject by Pulmonary Delivery of an Ethanol Metabolizing Enzyme-Use of ADH, ALDH, and NOX

The present example demonstrates an increase in the milligrams per liter of ethanol cleansed from human blood each minute following either (i) ethanol consumption (control; no enzyme in human lung); or (ii) ethanol consumption and subsequent inhalation of a nebulized solution comprising 100 mg alcohol dehydrogenase, 800 mg aldehyde dehydrogenase, and 100 mg NADH oxidase (enzyme in human lung).

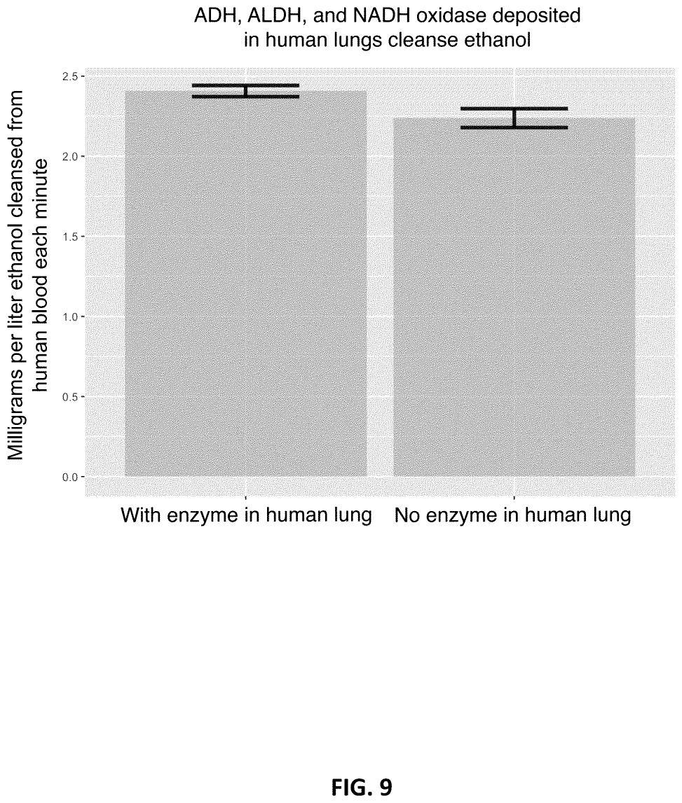

A single human subject subject completed each of conditions (i) and (ii) in two instances (day 1 condition (ii); day 3 condition (i); day 5 condition (ii); day 8 condition (i)). In brief, the human subject consumed 66.8 g of ethanol. After two hours, the same subject either did or did not inhale a nebulized solution comprising 100 mg alcohol dehydrogenase, 800 mg aldehyde dehydrogenase, and 100 mg NADH oxidase. Ethanol in the body was measured by drawing blood, purifying plasma, and subjecting that plasma to high performance liquid chromatography. Without inhaling the enzyme solution, ethanol was cleansed from the body at a measured rate of 2.237 milligrams per liter of ethanol each minute. After inhaling the enzyme solution, ethanol cleansing accelerated to a rate of 2.4056 milligrams per liter of ethanol each minute (FIG. 9).

These examples are provided for illustrative purposes only and not to limit the scope of the claims provided herein.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method of cleansing at least a portion of a lung of a subject comprising introducing to at least a portion of a lung of a subject a composition comprising at least one enzyme known to enzymatically breakdown at least one alcoholic irritant present systemically in said subject, including in at least a portion of said lung.

2. The method of claim 1, in which the at least one alcoholic irritant comprises methanol or ethanol.

3. The method of claim 2, in which said subject consumed one or more alcoholic beverages.

4. The method of claim 2, in which the at least one enzyme comprises alcohol dehydrogenase (ADH), aldehyde dehydrogenase (ALDH), variants thereof, or combinations thereof.

5. The method of claim 1, in which rate of cleansing the at least one alcoholic irritant from the subject's blood per minute is improved by at least about 1%, 2%, 3%, 4%, 5%, 6%, 7%, 8%, 9%, or 10% compared to a control subject wherein no enzyme known to enzymatically breakdown the least one alcoholic irritant was introduced.

6. A method of cleansing ethanol from at least a portion of a lung of a subject comprising introducing to at least a portion of a lung of a subject a composition comprising alcohol dehydrogenase (ADH), aldehyde dehydrogenase (ALDH), variants thereof, or combinations thereof.

7. The method of claim 6, in which said subject consumed one or more alcoholic beverages.

8. (canceled)

9. The method of claim 6, in which said composition is introduced immediately or shortly before, during, or immediately or shortly after the consumption of said one or more alcoholic beverages.

10. (canceled)

11. The method of claim 6, in which said subject's blood alcohol level is reduced by 10% or more, 25% or more, 50% or more, 75% or more, or 90% or more about one hour after said introduction compared to said subject's blood alcohol level in an absence of said introduction.

12. (canceled)

13. The method of claim 6, in which rate of cleansing ethanol from the subject's blood per minute is improved by at least about 1%, 2%, 3%, 4%, 5%, 6%, 7%, 8%, 9%, or 10% compared to a control subject wherein no ADH, ALDH, variants thereof, or combinations thereof were introduced.

14. A method of cleansing an alcoholic irritant from at least a portion of a lung of a subject comprising:
(a) introducing to at least a portion of a lung of a subject an effective amount of a composition comprising alcohol dehydrogenase (ADH), aldehyde dehydrogenase (ALDH), human variants thereof, or combinations thereof; and
(b) allowing an enzymatic breakdown of at least a portion of any alcoholic irritant that has diffused or migrated from the subject's circulatory system into the subject's lung and/or lung mucous.

15. The method of claim 14, in which said subject is contemplating a consumption of, is in a process of consuming, or has consumed one or more solid or liquid preparations comprising an alcoholic irritant.

16. The method of claim 15, in which said preparations comprises a pill, a capsule, a gummy, a sublingual, a lozenge, an injectable, a food, a beverage, or combinations thereof.

17. (canceled)

18. The method of claim 14, in which said composition further comprises coenzymes, cofactors, co-substrates, or combinations thereof.

19. (canceled)

20. The method of claim 14, in which said composition further comprises NADH oxidase.

21. The method of claim 14, in which rate of cleansing the alcoholic irritant from the subject's blood per minute is improved by at least about 1%, 2%, 3%, 4%, 5%, 6%, 7%, 8%, 9%, or 10% compared to a control subject wherein no ADH, ALDH, variants thereof, or combinations thereof were introduced.

22. The method of claim 20, in which rate of cleansing the alcoholic irritant from the subject's blood per minute is improved by at least about 1%, 2%, 3%, 4%, 5%, 6%, 7%, 8%, 9%, or 10% compared to a control subject wherein no ADH, ALDH, NADH oxidase, variants thereof, or combinations thereof were introduced.

23-25. (canceled)

26. The method of claim 1, in which said composition comprises a mist, an aerosol, a vapor, a drop, a snortable, an inhalable, a vapable, or a powder.

27. The method of claim 1, in which said enzyme, a variant thereof, or a combination thereof resides substantially in the subject's pulmonary tissue.

28-32. (canceled)

33. A method of freshening the breath of a subject comprising introducing to at least a portion of a lung of a subject a composition comprising at least one enzyme known to enzymatically breakdown at least one alcoholic irritant present systemically in said subject, including in at least a portion of said lung.

34-36. (canceled)
